(19) United States
(12) Patent Application Publication
(10) Pub. No.: US 2025/0197395 A1
(43) Pub. Date: Jun. 19, 2025

(54) IBOGAINE AND NORIBOGAINE ANALOGS AND USES THEREOF

(71) Applicant: atai Therapeutics, Inc., New York, NY (US)

(72) Inventors: Tanweer A. KHAN, Bridgewater, NJ (US); Glenn SHORT, Scituate, MA (US); Robert B. PERNI, Marlborough, MA (US); Alan C. GIBBS, Wyndmoor, PA (US); Jeffrey O'MEARA, Chertsey (CA); Harpreet KAUR, Brampton (CA); Ahmed Magdy ALI, Milton (CA)

(21) Appl. No.: 18/981,137

(22) Filed: Dec. 13, 2024

Related U.S. Application Data
(60) Provisional application No. 63/610,295, filed on Dec. 14, 2023.

(51) Int. Cl. C07D 471/08; A61K 31/439; A61K 31/55; C07D 471/22; C07D 491/048; C07D 491/22

(52) U.S. Cl. CPC C07D 471/08; A61K 31/439; A61K 31/55; C07D 471/22; C07D 491/048; C07D 491/22

(57) ABSTRACT

Provided herein are compounds of Formula (I), (II), (III), or pharmaceutically acceptable salt, prodrug, or stereoisomer thereof, and compositions thereof, wherein R₁, R₁′, R₂, R₂′, R₃, R₄, R₅, R₅′, R₆, R₇, R₉, R₉′, R₉″, X, and Z are defined herein. The disclosed compounds are useful for treating various conditions, including alcoholism, substance abuse disorder, and opioid use disorder

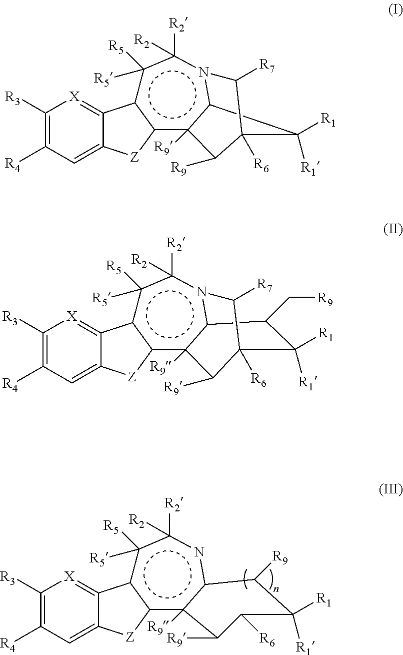

### CROSS-REFERENCE TO RELATED APPLICATION

This application claims the benefit of priority to U.S. Provisional Application No. 63/610,295, filed Dec. 14, 2023, which is hereby incorporated by reference in its entirety for all purposes.

### BACKGROUND OF THE INVENTION

Ibogaine is a naturally occurring psychoactive compound with anticipated therapeutic uses for the treatment of substance use disorders. In particular, recent animal research showed that ibogaine reduced self-administration of several drugs, including opiates, cocaine, and ethanol (Belgers et al., Transl Psychiatry, 6, e826 1-11, 2016). Ibogaine is metabolized in the body to its main metabolite, noribogaine, which is a non-hallucinogenic compound with an overlapping, but distinct profile of pharmacological effects.

The pharmacological basis for the therapeutic effects of ibogaine and noribogaine are unclear. Despite these benefits, the pharmacokinetic and biodistribution profile of ibogaine potentially limits its therapeutic utility.

There is a need for improved derivatives of ibogaine and noribogaine.

### BRIEF SUMMARY OF THE INVENTION

In embodiments, the present disclosure provides a compound of Formula (I):

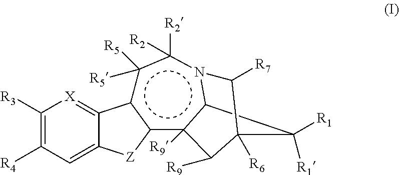

or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ, —C(═O)ORₐ, —CONHRₐ, —CONRₐRₐ, —CH₂ORₐ, or R₁ and R₁′ taken together with the atom to which they are attached form ═O, ═S, ═NH, ═NOH, or ═NORₐ;
R₂ and R₂′ are independently absent, hydrogen, deuterium, optionally substituted alkyl (e.g., deuterated alkyl or halogenated alkyl), optionally substituted aryl, optionally substituted heteroalkyl, —ORₐ-alkylene-ORₐ—CH₂C(═O)ORₐ, —CH₂C(═O)NRₐRₐ or R₂ and R₂′ taken together with the atom to which they are attached form ═O, ═S, ═NH, ═NOH, or ═NORₐ;
each R₃ and R₄ is independently hydrogen, deuterium, —ORₐ, halogen, —NO₂, —CN, —NRₐRₐ, —SRₐ, —SORₐ, —SO₂Rₐ, —C(═O)ORₐ, —C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl (e.g., deuterated alkyl and halogenated alkyl), fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, -alkylene-ORₐ—C(═O)ORₐ or —C(═O)NRₐRₐ;
R₇, R₉, and R₉′ are independently hydrogen, deuterium, optionally substituted alkyl (e.g., deuterated alkyl and halogenated alkyl), fluoro, —ORₐ, —C(═O)ORₐ, —CH₂ORₐ, —CH₂SRₐ, —CH₂NRₐRₐ, aryl, heteroaryl, or cycloalkyl;
each Rₐ is independently hydrogen, alkyl, deuterated alkyl, alkenyl, alkylene-aryl, alkylene-cycloalkyl, aryl, or heteroaryl;
Z is O, S, or N(R₈), wherein R₈ is hydrogen, deuterium, alkyl, deuterated alkyl, —CH₂—OH, —CH₂O—Rₐ, —CH₂SH, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ.

In embodiments, the present disclosure provides a compound of Formula (II):

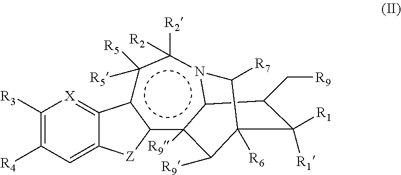

or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ—C(═O)ORₐ—CH₂ORₐ, or R₁ and R₁′ taken together with the atom to which they are attached form ═NH, ═NOH, or ═NORₐ;
R₂ and R₂′ are independently absent, hydrogen, deuterium, optionally substituted alkyl (e.g., deuterated alkyl or halogenated alkyl), optionally substituted aryl, optionally substituted heteroalkyl, —ORₐ-alkylene-ORₐ—CH₂C(═O)ORₐ, or —CH₂C(═O)NRₐRₐ;
each R₃ and R₄ is independently hydrogen, deuterium, —ORₐ, halogen, —NO₂, —CN, —NRₐRₐ, —SRₐ, —SORₐ, —SO₂Rₐ, —C(═O)ORₐ, —C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl (e.g., deuterated alkyl and halogenated alkyl), fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, alkylene-ORₐ, —C(═O)ORₐ, or —C(═O)NRₐRₐ;
R₇ is hydrogen, deuterium, optionally substituted alkyl (e.g., deuterated alkyl and halogenated alkyl), fluoro, —ORₐ, —C(═O)ORₐ, —CH₂ORₐ, —CH₂SRₐ, —CH₂NRₐRₐ, aryl, heteroaryl, or cycloalkyl;
R₉, R₉′ and R₉″ are independently hydrogen, deuterium, optionally substituted alkyl, fluoro, —ORₐ, —C(═O)ORₐ, —CH₂ORₐ, —CH₂SRₐ, —CH₂NRₐRₐ, aryl, heteroaryl, or cycloalkyl
each Rₐ is independently hydrogen, alkyl, deuterated alkyl, alkenyl, alkylene-aryl, alkylene-cycloalkyl, aryl, or heteroaryl;
Z is O, S, or N(R₈), wherein R₈ is hydrogen, deuterium, alkyl, deuterated alkyl, —CH₂—OH, —CH₂O—Rₐ, —CH₂SH, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ,
wherein at least one of R₂, R₂′, R₅, R₅′, or R₇ is not hydrogen, and
wherein the compound is not

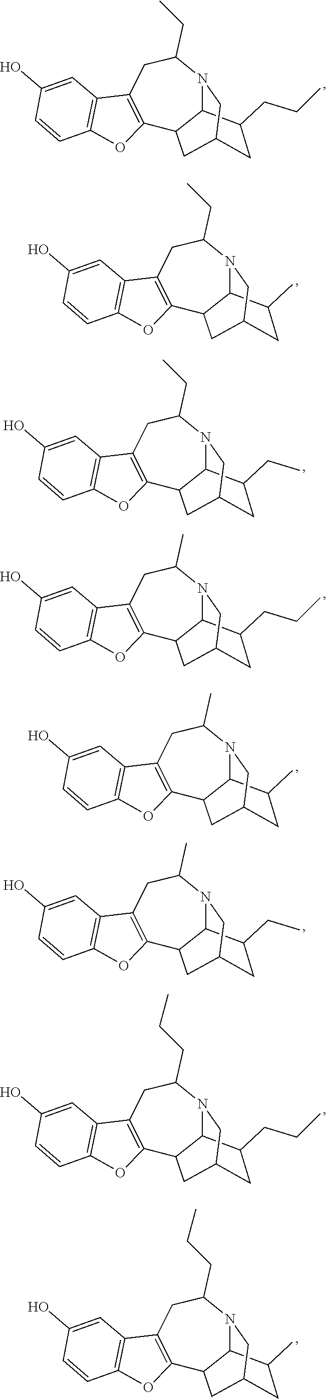

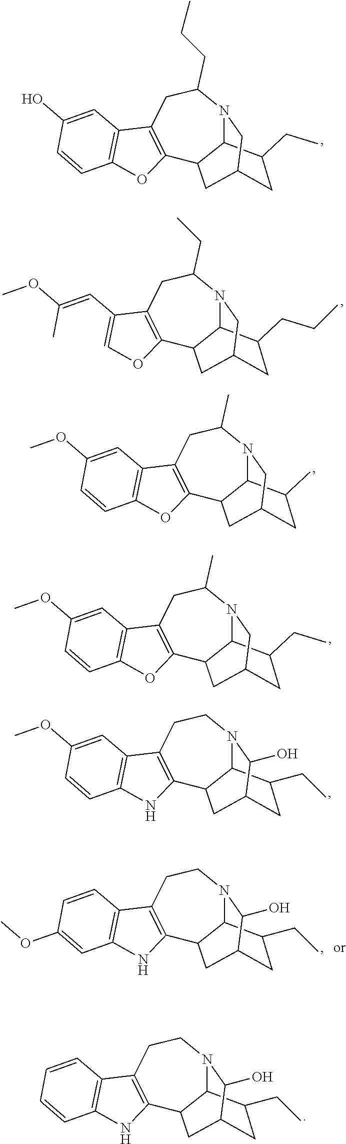

In embodiments, the present disclosure provides a compound of Formula (II):

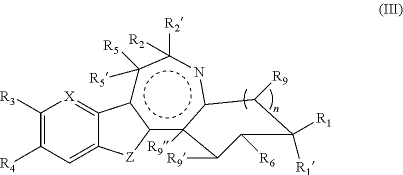

or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ—C(═O)ORₐ—CH₂ORₐ, or R₁ and R₁′ taken together with the atom to which they are attached form ═NH, ═NOH, or ═NORₐ;
R₂ and R₂′ are independently absent, hydrogen, deuterium, optionally substituted alkyl (e.g., deuterated alkyl or halogenated alkyl), optionally substituted aryl, optionally substituted heteroalkyl, —ORₐ-alkylene-ORₐ, —CH₂C(═O)ORₐ, —CH₂C(═O)NRₐRₐ or R₂ and R₂′ taken together with the atom to which they are attached form ═NH, ═NOH, or ═NORₐ;
each R₃ and R₄ is independently hydrogen, deuterium, —ORₐ, halogen, —NO₂, —CN, —NRₐRₐ, —SRₐ, —C(═O)ORₐ—C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl (e.g., deuterated alkyl and halogenated alkyl), fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, alkylene-ORₐ, —C(═O)ORₐ or —C(═O)NRₐRₐ;
R₉, R₉′, and R₉″ are independently hydrogen, deuterium, optionally substituted alkyl, fluoro, —CN, —ORₐ, —C(═O)ORₐ—C(═O)NRₐRₐ, —CH₂ORₐ, —CH₂SRₐ, —CH₂NRₐRₐ, aryl, heteroaryl, or cycloalkyl
each Rₐ is independently hydrogen, alkyl, deuterated alkyl, alkenyl, alkylene-aryl, alkylene-cycloalkyl, aryl, or heteroaryl;
n is 0, 1 or 2; and
Z is O, S, or N(R₈), wherein R₈ is hydrogen, deuterium, alkyl, deuterated alkyl, —CH₂—OH, —CH₂O—Rₐ, —CH₂SH, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ.

### DETAILED DESCRIPTION OF THE INVENTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications (including non-patent publications) are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

The terms “administer,” “administering” or “administration” as used herein refer to either directly administering a compound or a composition comprising the compound to a patient.

The term “treating” as used herein with regard to a patient, refers to improving at least one symptom of the patient's disorder. In embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient in need thereof.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris -(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈alkyl. A C₁-C₈alkyl includes C₅ alkyls, C₄ alkyls, C₃ alkyls, C₂ alkyls and C₁ alkyl (i.e., methyl). A C₁-C₆ alkyl includes all moieties described above for C₁-C₈alkyls but also includes C₆ alkyls. A C₁-C₁₀ alkyl includes all moieties described above for C₁-C₈alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, 1-butyl, n-pentyl, t-amyl, n-hexyl, n-heptyl, n-octyl, n-nonyl, n-decyl, n-undecyl, and n-dodecyl. Unless stated otherwise, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise, an alkylene chain can be optionally substituted.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl. Unless stated otherwise, an alkenyl group can be optionally substituted.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups. Unless stated otherwise, an alkynyl group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise, the “aryl” can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, including compounds with aromatic resonance structures (e.g., 2-pyridone), and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzofuranyl, benzooxazolyl, benzothiadiazolyl, benzo[b][1,4]dioxepinyl, 1,4 benzodioxanyl, benzonaphthofuranyl, benzoxazolyl, benzodioxolyl, benzodioxinyl, benzopyranyl, benzopyranonyl, benzofuranonyl, benzothienyl(benzothiophenyl), benzotriazolyl, benzo[4,6]imidazo[1,2a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2 oxoazepinyl, oxazolyl, oxiranyl, 1-oxidopyridinyl, 1 oxidopyrimidinyl, 1-oxidopyrazinyl, 1-oxidopyridazinyl, 1 phenyl 1H pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise, a heteroaryl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated, a cycloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Haloalkyl” or “halogenated alkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise, a haloalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double-or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O) Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O) R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, N-heterocyclyl, heterocyclylalkyl, heteroaryl, N-heteroaryl and/or heteroarylalkyl group. In some embodiments, “substituted” further means any alkyl, cycloalkyl or heterocyclylalkyl in which one or more hydrogen atoms is replaced by an isotope e.g., deuterium.

### Compounds

The present disclosure provides compounds that are analogs of ibogaine and noribogaine as well as pharmaceutical compositions thereof and uses thereof in treating various diseases and disorders.

In embodiments, the present disclosure provides a compound of Formula (I):

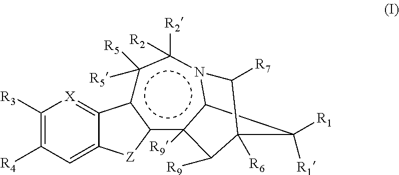

or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ, —C(═O)ORₐ, —CONHRₐ, —CONRₐRₐ, —CH₂ORₐ, or R₁ and R₁′ taken together with the atom to which they are attached form ═O, ═S, ═NH, ═NOH, or ═NORₐ;
R₂ and R₂′ are independently absent, hydrogen, deuterium, optionally substituted alkyl (e.g., deuterated alkyl or halogenated alkyl), optionally substituted aryl, optionally substituted heteroalkyl, —ORₐ-alkylene-ORₐ—CH₂C(═O)ORₐ, —CH₂C(═O)NRₐRₐ or R₂ and R₂′ taken together with the atom to which they are attached form ═O, ═S, ═NH, ═NOH, or ═NORₐ;
each R₃ and R₄ is independently hydrogen, deuterium, —ORₐ, halogen, —NO₂, —CN, —NRₐRₐ, —SRₐ, —SORₐ, —SO₂Rₐ, —C(═O)ORₐ, —C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl (e.g., deuterated alkyl and halogenated alkyl), fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, alkylene-ORₐ, —C(═O)ORₐ—C(═O)NRₐRₐ;
R₇, R₉, and R₉′ are independently hydrogen, deuterium, optionally substituted alkyl (e.g., deuterated alkyl and halogenated alkyl), fluoro, —ORₐ, —C(═O)ORₐ, —CH₂ORₐ—CH₂SRₐ, —CH₂NRₐRₐ, aryl, heteroaryl, or cycloalkyl;
each Rₐ is independently hydrogen, alkyl, deuterated alkyl, alkenyl, alkylene-aryl, alkylene-cycloalkyl, aryl, or heteroaryl;
Z is O, S, or N(R₈), wherein R₈ is hydrogen, deuterium, alkyl, deuterated alkyl, —CH₂—OH, CH₂O—Rₐ, —CH₂SH, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ.

In embodiments of the compound of Formula (I), X is N. In embodiments, X is C(R₃).

In embodiments, Z is O. In embodiments, Z is N(R₈).

In embodiments, R₁ and R₁′ are independently hydrogen, —ORₐ, or R₁ and R₁′ taken together with the atom to which they are attached form ═O. In embodiments, R₁ and R₁′ are independently hydrogen, —OCH₃ or —OH.

In embodiments, R₂ and R₂′ are independently hydrogen, optionally substituted alkyl, or -alkylene-ORₐ. In embodiments, R₂ and R₂′ are hydrogen. In embodiments, R₂ and R₂′ are independently hydrogen or —CH₂—OH.

In embodiments, R₃ is —OH. In embodiments, R₃ is —OCH₃.

In embodiments, R₄ is hydrogen. In embodiments, R₄ is —OH. In embodiments, R₄ is —OCH₃.

In embodiments, R₃ and R₄ are —OH. In embodiments, R₃ and R₄ are —OCH₃.

In embodiments, R₅ and R₅' are independently hydrogen, —ORₐ, or fluoro. In embodiments, R₅ and R₅' are independently hydrogen, or —OH. In embodiments, R₅ and R₅' are fluoro.

In embodiments, R₆ is hydrogen.

In embodiments, R₇ is hydrogen. In embodiments, R₇ is optionally substituted alkyl or —CH₂ORₐ. In embodiments, R₇ is —CN. In embodiments, R₇ is —CH₂OH.

In embodiments, R₉ is hydrogen.

In embodiments, R₉′ is hydrogen.

In embodiments, the compound of Formula (I) is a compound of Formula (Ia) or a pharmaceutically acceptable salt, prodrug, or stereoisomer thereof:

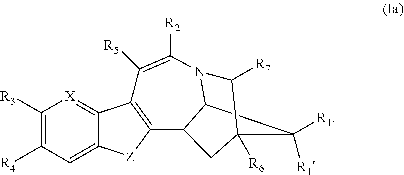

In embodiments of the compounds of Formula (I) or (Ia), the compound is selected from:

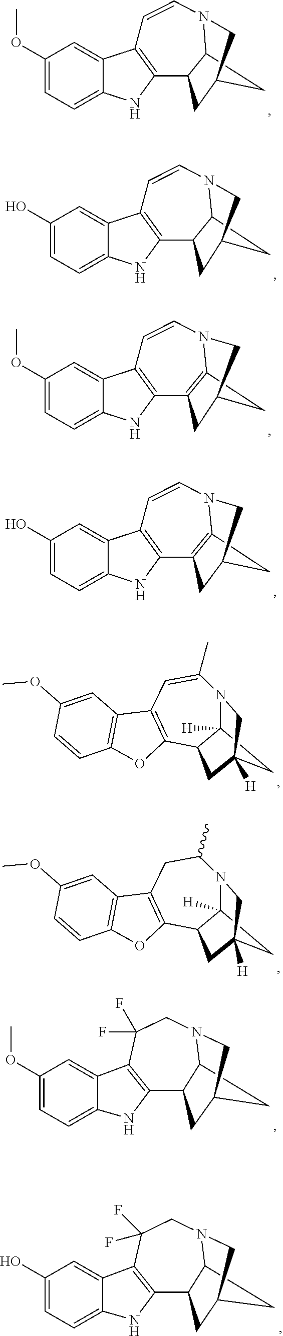

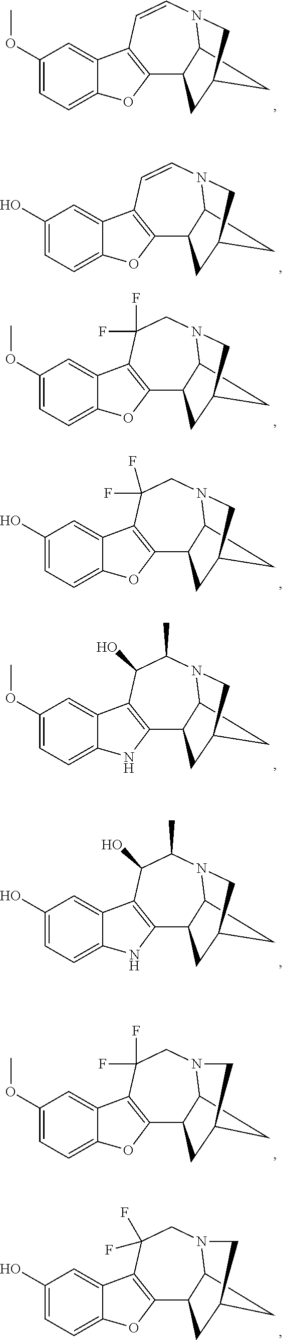

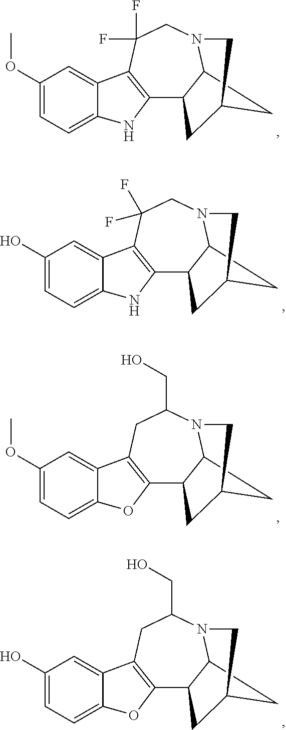 [SMILES: COc1ccc2[nH]c3c(c2c1)C(F)(F)CN1C[C@H]2CC1[C@H]3C2 ; Oc1ccc2[nH]c3c(c2c1)C(F)(F)CN1C[C@H]2CC1[C@H]3C2 ; COc1ccc2oc3c(c2c1)CC(CO)N1C[C@H]2CC1[C@H]3C2 ; OCC1Cc2c(oc3ccc(O)cc23)[C@@H]2C[C@@H]3CC2N1C3]

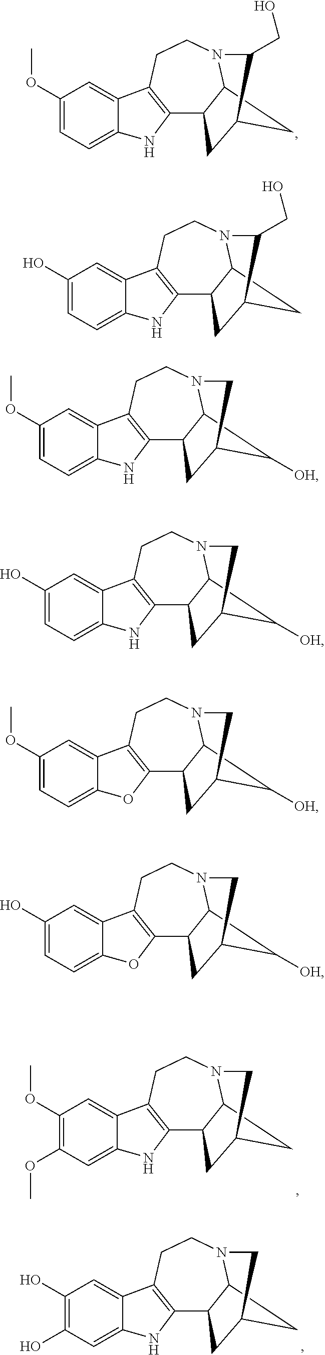

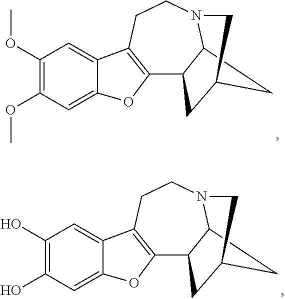 [SMILES: COc1cc2oc3c(c2cc1OC)CCN1C[C@H]2CC1[C@H]3C2 ; Oc1cc2oc3c(c2cc1O)CCN1C[C@H]2CC1[C@H]3C2]

or a pharmaceutically acceptable salt, prodrug, or stereoisomer thereof.

In embodiments, the present disclosure provides a compound of Formula (II):

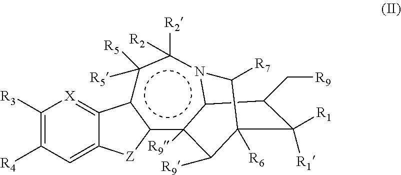

or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ, —C(═O)ORₐ, —CH₂OR, or R₁ and R₁′ taken together with the atom to which they are attached form ═NH, ═NOH, or ═NORₐ;
R₂ and R₂′ are independently absent, hydrogen, deuterium, optionally substituted alkyl (e.g., deuterated alkyl or halogenated alkyl), optionally substituted aryl, optionally substituted heteroalkyl, —ORₐ alkylene-ORₐ, —CH₂C(═O)ORₐ, or —CH₂C(═O)NRₐRₐ;
each R₃ and R₄ is independently hydrogen, deuterium, —ORₐ, halogen, —NO₂, —CN, —NRₐRₐ, —SRₐ, —SORₐ, —SO₂Rₐ, —C(═O)ORₐ, —C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl (e.g., deuterated alkyl and halogenated alkyl), fluoro, —ORₐ, —C(═O)ORₐ, —CH₂ORₐ, —CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, alkylene-ORₐ, —C(═O)ORₐ—C(═O)NRₐRₐ;
R₇ is hydrogen, deuterium, optionally substituted alkyl (e.g., deuterated alkyl and halogenated alkyl), fluoro, —ORₐ, —C(═O)ORₐ, —CH₂ORₐ, —CH₂SRₐ, —CH₂NRₐRₐ, aryl, heteroaryl, or cycloalkyl;
R₉, R₉′ and R₉″ are independently hydrogen, deuterium, optionally substituted alkyl, fluoro, —ORₐ, —C(═O)ORₐ, —CH₂ORₐ—CH₂SRₐ, —CH₂NRₐRₐ, aryl, heteroaryl, or cycloalkyl each Rₐ is independently hydrogen, alkyl, deuterated alkyl, alkenyl, alkylene-aryl, alkylene-cycloalkyl, aryl, or heteroaryl;
Z is O, S, or N(R₈), wherein R₈ is hydrogen, deuterium, alkyl, deuterated alkyl, —CH₂—OH, CH₂O—Rₐ, —CH₂SH, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ.
wherein at least one of R₂, R₂′, R₅, R₅' or R₇ is not hydrogen.

In embodiments of compound of Formula (II), the compound is not

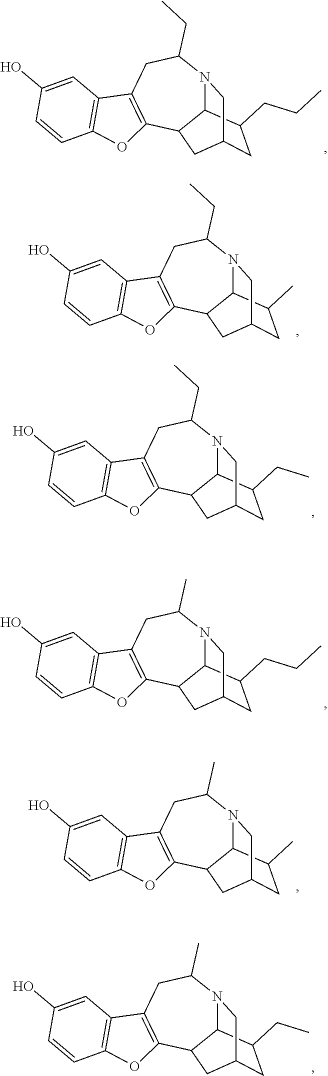

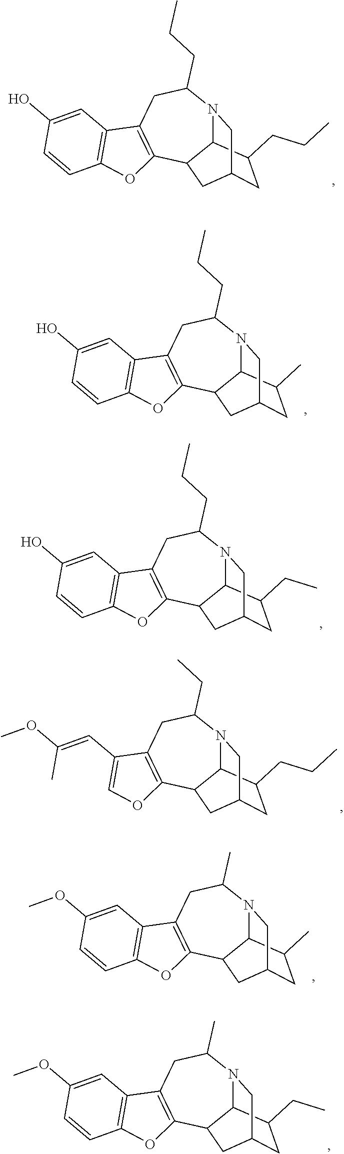

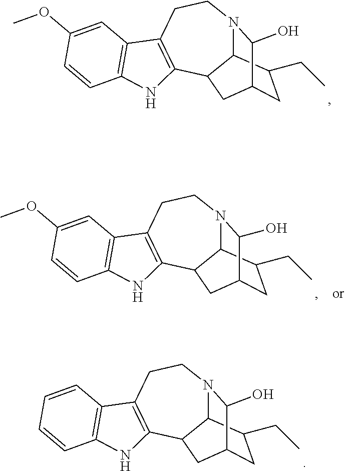

In embodiments of the compound of Formula (II), X is N. In embodiments, X is C(R₃).

In embodiments, Z is O. In embodiments, Z is N(R₈).

In embodiments, R₁ and R₁′ are independently hydrogen.

In embodiments, R₂ and R₂′ are independently hydrogen, optionally substituted alkyl, or -alkylene-ORₐ. In embodiments, R₂ and R₂′ are hydrogen. In embodiments, R₂ and R₂′ are independently hydrogen or —CH₂—OH.

In embodiments, R₃ is —OH. In embodiments, R₃ is —OCH₃.

In embodiments, R₄ is hydrogen. In embodiments, R₄ is —OH. In embodiments, R₄ is —OCH₃.

In embodiments, R₃ and R₄ are —OH. In embodiments, R₃ and R₄ are —OCH₃.

In embodiments, R₅ and R₅ are independently hydrogen, —ORₐ, or fluoro. In embodiments, R₅ and R₅ are independently hydrogen, or —OH. In embodiments, R₅ and R₅ are fluoro.

In embodiments, R₆ is hydrogen.

In embodiments, R₇ is hydrogen. In embodiments, R₇ is optionally substituted alkyl or —CH₂ORₐ. In embodiments, R₇ is —CH₂OH.

In embodiments, R₉ is —CH₃. In embodiments, R₉ is hydrogen.

In embodiments, R₉′ is hydrogen.

In embodiments, R₉″ is hydrogen.

In embodiments, the compound of Formula (II) is a compound of Formula (IIa) or a pharmaceutically acceptable salt, prodrug, or stereoisomer thereof:

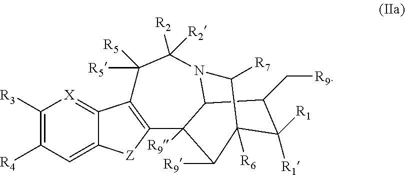

In embodiments of the compounds of Formula (II), the compound is selected from:

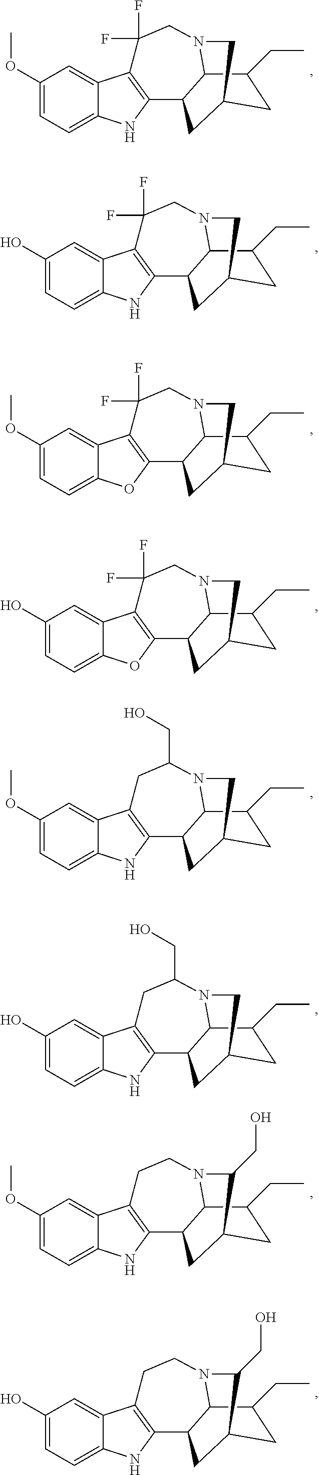

or a pharmaceutically acceptable salt, prodrug, or stereoisomer thereof.

In embodiments, the present disclosure provides a compound of Formula (III):

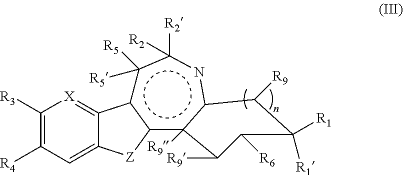

or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ, —C(═O)ORₐ, —CH₂ORₐ or R₁ and R₁′ taken together with the atom to which they are attached form ═NH, ═NOH, or ═NORₐ;
R₂ and R₂′ are independently absent, hydrogen, deuterium, optionally substituted alkyl (e.g., deuterated alkyl or halogenated alkyl), optionally substituted aryl, optionally substituted heteroalkyl, —ORₐ, alkylene-ORₐ—CH₂C(═O)ORₐ, —CH₂C(═O)NRₐRₐ, or R₂ and R₂′ taken together with the atom to which they are attached form ═NH, ═NOH, or ═NORₐ;
each R₃ and R₄ is independently hydrogen, deuterium, —ORₐ, halogen, —NO₂, —CN, —NRₐRₐ, —SRₐ, —C(═O)ORₐ—C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl (e.g., deuterated alkyl and halogenated alkyl), fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, alkylene-ORₐ, —C(═O)ORₐ—C(═O)NRₐRₐ;
R₉, R₉′, and R₉″ are independently hydrogen, deuterium, optionally substituted alkyl, fluoro, —CN, —ORₐ, —C(═O)ORₐ, —C(═O)NRₐRₐ, —CH₂ORₐ—CH₂SRₐ, —CH₂NRₐRₐ, aryl, heteroaryl, or cycloalkyl
each Rₐ is independently hydrogen, alkyl, deuterated alkyl, alkenyl, alkylene-aryl, alkylene-cycloalkyl, aryl, or heteroaryl;
n is 0, 1 or 2; and
Z is O, S, or N(R₈), wherein R₈ is hydrogen, deuterium, alkyl, deuterated alkyl, —CH₂—OH, —CH₂O—Rₐ, —CH₂SH, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ.

In embodiments of the compound of Formula (III), X is N. In embodiments, X is C(R₃).

In embodiments, Z is O. In embodiments, Z is N(R₈).

In embodiments, R₁ and R₁′ are independently hydrogen.

In embodiments, R₂ and R₂′ are independently hydrogen, optionally substituted alkyl, or -alkylene-ORₐ. In embodiments, R₂ and R₂′ are hydrogen. In embodiments, R₂ and R₂′ are independently hydrogen or —CH₂—OH.

In embodiments, R₃ is —OH. In embodiments, R₃ is —OCH₃.

In embodiments, R₄ is hydrogen. In embodiments, R₄ is —OH. In embodiments, R₄ is —OCH₃. In embodiments, R₃ and R₄ are —OH. In embodiments, R₃ and R₄ are —OCH₃.

In embodiments, R₅ and R₅' are independently hydrogen, —ORₐ, or fluoro. In embodiments, R₅ and R₅' are independently hydrogen, or —OH. In embodiments, R₅ and R₅' are fluoro.

In embodiments, R₆ is hydrogen.

In embodiments, R₉ is hydrogen. In embodiments, R₉′ is hydrogen. In embodiments, R₉″ is hydrogen.

In embodiments of the compounds of Formula (III), the compound is

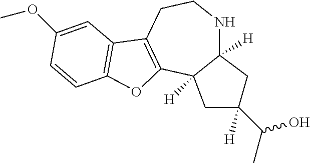 [SMILES: COc1ccc2oc3c(c2c1)CCN[C@H]1C[C@@H](C(C)O)C[C@@H]31]

or a pharmaceutically acceptable salt, prodrug, or stereoisomer thereof.

In embodiments of the compounds of Formula (I), (Ia), (II), (IIa), or (III), X is N. In embodiments, X is C(R₃). In embodiments, X is C(R₃) and R₃ is hydrogen. In embodiments, X is C(R₃) and R₃ is deuterium. In embodiments, X is C(R₃) and R₃ is —ORₐ. In embodiments, R₃ is —OH. In embodiments, R₃ is —OCH₃. In embodiments, R₃ is —OCH₂CH₃. In embodiments, X is C(R₃) and R₃ is halogen. In embodiments, X is C(R₃) and R₃ is —F. In embodiments, X is C(R₃) and R₃ is —NO₂. In embodiments, X is C(R₃) and R₃ is —CN. In embodiments, X is C(R₃) and Rₐ is —NRₐRₐ. In embodiments, R₃ is —NH₂. In embodiments, X is C(R₃) and R₃ is —SRₐ. In embodiments, R₃ is —SH. In embodiments, X is C(R₃) and R₃ is —C(═O)ORₐ. In embodiments, R₃ is —C(═O)OH. In embodiments, X is C(R₃) and R₃ is —C(═O)NRₐRₐ. In embodiments, R₃ is —C(═O)NH₂. In embodiments, X is C(R₃) and R₃ is —OAc. In embodiments, X is C(R₃) and R₃ is —CH₂ORₐ. In embodiments, R₃ is —CH₂OH. In embodiments, X is C(R₃) and R₃ is —CH₂SRₐ. In embodiments, R₃ is —CH₂SH. In embodiments, X is C(R₃) and R₃ is —CH₂NRₐRₐ. In embodiments, R₃ is —CH₂NH₂.

In embodiments of the compounds of Formula (I), (Ia), (II), (IIa), or (III), Z is O. In embodiments, Z is S. In embodiments, Z is N(R₈). In embodiments, Z is N(R₈) and Rₐ is hydrogen. In embodiments, Z is N(R₈) and Rₐ is deuterium. In embodiments, Z is N(R₈) and Rₐ is alkyl. In embodiments, R₈ is CH₃. In embodiments, Rₐ is Ch₂CH₃. In embodiments, Z is N(R₈) and Rₐ is —CH₂OH. In embodiments, Z is N(R₈) and Rₐ is —CH₂O—Rₐ. In embodiments, Rₐ is —CH₂OCH₃. In embodiments, R₈ is —CH₂OCH₂CH₃. In embodiments, Z is N(R₈) and Rₐ is —CH₂SH. In embodiments, Z is N(R₈) and Rₐ is —CH₂S—Rₐ. In embodiments, Rₐ is —CH₂SCH₃. In embodiments, Z is N(R₈), and R₈ is —CH₂NRₐRₐ. In embodiments, Rₐ is —CH₂NH₂. In embodiments, Rₐ is —CH₂N(CH₃)₂. In embodiments, Z is N(R₈), and Rₐ is —C(═O)ORₐ. In embodiments, Z is N(R₈), and Rₐ is —COOH. In embodiments, Rₐ is —COOCH₃. In embodiments, Z is N(R₈), and Rₐ is —CONHRₐ. In embodiments, Rₐ is —CONH₂. In embodiments, Rₐ is —CONHCH₃. In embodiments, Z is N(R₈), and Rₐ is —CONRₐRₐ. In embodiments, Rₐ is —CON(CH₃)₂. In embodiments, Rₐ is —P(═O)(OH)₂. In embodiments, R₈ is —CH(Rₐ)OC(═O)ORₐ. In embodiments, R₈ is —CH(Rₐ)OP(═O)(ORₐ)₂. In embodiments, Rₐ is —SO₂Rₐ.

In embodiments of the compounds of Formula (I), (Ia), (II), (IIa), or (III), R₁ is hydrogen. In embodiments, R₁ is deuterium. In embodiments, R₁ is fluoro. In embodiments, R₁ is optionally substituted alkyl. In embodiments, R₁ is —C₁-C₆ alkyl (for example, C₁, C₂, C₃, C₄, C₆, or C₅). In embodiments, R₁ is —CH₃. In embodiments, R₁ is —CH₂CH₃. In embodiments, R₁ is —CH₂ORₐ. In embodiments, R₁ is —CH₂OH. In embodiments, R₁ is —CH₂OCH₃. In embodiments, R₁ is —ORₐ. In embodiments, R₁ is —OH. In embodiments, R₁ is —OCH₃. In embodiments, R₁ is —OCH₂CH₃.

In embodiments of the compounds of Formula (I), (Ia), (II), (IIa), or (III), R₁′ is hydrogen. In embodiments, R₁′ is deuterium. In embodiments, R₁′ is fluoro. In embodiments, R₁′ is optionally substituted alkyl. In embodiments, R₁′ is —C₁-C₆ alkyl (for example, C₁, C₂, C₃, C₄, C₆, or C₅). In embodiments, R₁′ is —CH₃. In embodiments, R₁′ is —CH₂CH₃. In embodiments, R₁′ is —CH₂ORₐ. In embodiments, R₁′ is —CH₂OH. In embodiments, R₁′ is —CH₂OCH₃. In embodiments, R₁′ is —ORₐ. In embodiments, R₁′ is —OH. In embodiments, R₁′ is —OCH₃. In embodiments, R₁′ is —OCH₂CH₃.

In embodiments, R₁ and R₁′ taken together with the atom to which they are attached form ═O.

In embodiments of the compounds of Formula (I), (Ia), (II), (IIa), or (III), R₂ is absent. In embodiments, R₂ is hydrogen. In embodiments, R₂ is deuterium. In embodiments, R₂ is optionally substituted alkyl. In embodiments, R₂ is CH₃. In embodiments, R₂ is CH₂CH₃. In embodiments, R₂ is deuterated alkyl. In embodiments, R₂ is halogenated alkyl. In embodiments, R₂ is optionally substituted aryl. In embodiments, R₂ is optionally substituted heteroalkyl. In embodiments, R₂ is —ORₐ. In embodiments, R₂ is —OH. In embodiments, R₂ is -alkylene-ORₐ. In embodiments, R₂ is —CH₂—OH. In embodiments, R₂ is —CH₂CH₂—OH. In embodiments, R₂ is —CH₂C(═O)ORₐ. In embodiments, R₂ is —CH₂C(═O)OH. In embodiments, R₂ is —CH₂C(═O)NRₐRₐ. In embodiments, R₂ is —CH₂C(═O)NH₂.

In embodiments of the compounds of Formula (I), (II), (IIa), or (III), R₂′ is absent. In embodiments, R₂′ is hydrogen. In embodiments, R₂′ is deuterium. In embodiments, R₂′ is optionally substituted alkyl. In embodiments, R₂′ is CH₃. In embodiments, R₂′ is CH₂CH₃. In embodiments, R₂′ is deuterated alkyl. In embodiments, R₂′ is halogenated alkyl. In embodiments, R₂′ is optionally substituted aryl. In embodiments, R₂′ is optionally substituted heteroalkyl. In embodiments, R₂′ is —ORₐ. In embodiments, R₂′ is —OH. In embodiments, R₂′ is -alkylene-ORₐ. In embodiments, R₂′ is —CH₂—OH. In embodiments, R₂′ is —CH₂CH₂—OH. In embodiments, R₂′ is —CH₂C(═O)ORₐ. In embodiments, R₂′ is —CH₂C(═O)OH. In embodiments, R₂′ is —CH₂C(═O)NRₐRₐ. In embodiments, R₂′ is —CH₂C(═O)NH₂.

In embodiments of the compounds of Formula (I), (Ia), (II), (IIa), or (III), R₃ is hydrogen. In embodiments, R₃ is deuterium. In embodiments, R₃ is halogen. In embodiments, R₃ is —F. In embodiments, R₃ is —Cl. In embodiments, R₃ is —Br. In embodiments, R₃ is —I. In embodiments, R₃ is —ORₐ. In embodiments, R₃ is —OH. In embodiments, R₃ is —OCH₃. In embodiments, R₃ is —OCH₂CH₃. In embodiments, R₃ is —NO₂. In embodiments, R₃ is —CN. In embodiments, R₃ is —NRₐRₐ. In embodiments, R₃ is —NH₂. In embodiments, R₃ is —NH (alkyl). In embodiments, R₃ is —NH (alkenyl). In embodiments, R₃ is —NH (alkynyl). In embodiments, R₃ is —NH (aryl). In embodiments, R₃ is —NH (heteroaryl). In embodiments, R₃ is —N(cycloalkyl). In embodiments, R₃ is —SRₐ. In embodiments, R₃ is —SH. In embodiments, R₃ is —C(═O)ORₐ. In embodiments, R₃ is —C(═O)OH. In embodiments, R₃ is —C(═O)NRₐRₐ. In embodiments, R₃ is —C(═O)NH₂. In embodiments, R₃ is —C(═O)NRₐRₐ. In embodiments, R₃ is —OAc. In embodiments, R₃ is —CH₂ORₐ. In embodiments, R₃ is —CH₂OH. In embodiments, R₃ is —CH₂SRₐ. In embodiments, R₃ is —CH₂SH. In embodiments, R₃ is —CH₂NRₐRₐ. In embodiments, R₃ is —CH₂NH₂.

In embodiments of the compounds of Formula (I), (Ia), (II), (IIa), or (III), R₄ is hydrogen. In embodiments, R₄ is deuterium. In embodiments, R₄ is halogen. In embodiments, R₄ is —F. In embodiments, R₄ is —Cl. In embodiments, R₄ is —Br. In embodiments, R₄ is —I. In embodiments, R₄ is —ORₐ. In embodiments, R₄ is —OH. In embodiments, R₄ is —OCH₃. In embodiments, R₄ is —OCH₂CH₃. In embodiments, R₄ is —NO₂. In embodiments, R₄ is —CN. In embodiments, R₄ is —NRₐRₐ. In embodiments, R₄ is —NH₂. In embodiments, R₄ is —NH (alkyl). In embodiments, R₄ is —NH (alkenyl). In embodiments, R₄ is —NH (alkynyl). In embodiments, R₄ is —NH (aryl). In embodiments, Rₐ is —NH (heteroaryl). In embodiments, R₄ is —N(cycloalkyl). In embodiments, R₄ is —SRₐ. In embodiments, R₄ is —SH. In embodiments, R₄ is —C(═O)ORₐ. In embodiments, R₄ is —C(═O)OH. In embodiments, R₄ is —C(═O)NRₐRₐ. In embodiments, R₄ is —C(═O)NH₂. In embodiments, R₄ is —C(═O)NRₐRₐ. In embodiments, R₄ is —OAc. In embodiments, R₄ is —CH₂ORₐ. In embodiments, R₄ is —CH₂OH. In embodiments, R₄ is —CH₂SRₐ. In embodiments, R₄ is —CH₂SH. In embodiments, R₄ is —CH₂NRₐRₐ. In embodiments, R₄ is —CH₂NH₂.

In embodiments, R₃ and R₄ are hydrogen. In embodiments, R₃ is hydrogen and R₄ is —OH. In embodiments, R₃ is —OH and R₄ is hydrogen. In embodiments, R₃ and R₄ are —OH. In embodiments, R₃ is hydrogen and R₄ is —OCH₃. In embodiments, R₃ is —OCH₃ and Rₐ is hydrogen. In embodiments, R₃ and R₄ are —OCH₃.

In embodiments of the compounds of Formula (I), (Ia), (II), (IIa), or (III), R₅ is absent. In embodiments, R₅ is hydrogen. In embodiments, R₅ is deuterium. In embodiments, R₅ is optionally substituted alkyl. In embodiments, R₅ is CH₃. In embodiments, R₅ is CH₂CH₃. In embodiments, R₅ is deuterated alkyl. In embodiments, R₅ is halogenated alkyl. In embodiments, R₅ is fluoro. In embodiments, R₅ is —ORₐ. In embodiments, R₅ is —OH. In embodiments, R₅ is —OCH₃. In embodiments, R₅ is —OCH₂CH₃. In embodiments, R₅ is C(═O)ORₐ. In embodiments, R₅ is C(═O)OH. In embodiments, R₅ is —CH₂ORₐ. In embodiments, R₅ is —CH₂OH. In embodiments, R₅ is —CH₂SRₐ. In embodiments, R₅ is —CH₂SH. In embodiments, R₅ is —CH₂NRₐRₐ. In embodiments, R₅ is —CH₂NH₂. In embodiments, R₂ is —CH₂NHCH₃. In embodiments, R₅ is aryl. In embodiments, R₅ is heteroaryl. In embodiments, R₅ is cycloalkyl.

In embodiments of the compounds of Formula (I), (II), (IIa), or (III), R₅' is absent. In embodiments, R₅' is hydrogen. In embodiments, R₅' is deuterium. In embodiments, R₅' is optionally substituted alkyl. In embodiments, R₅' is CH₃. In embodiments, R₅' is CH₂CH₃. In embodiments, R₅' is deuterated alkyl. In embodiments, R₅' is halogenated alkyl. In embodiments, R₅' is fluoro. In embodiments, R₅' is —ORₐ. In embodiments, R₅' is —OH. In embodiments, R₅' is —OCH₃. In embodiments, R₅' is —OCH₂CH₃. In embodiments, R₅' is C(═O)ORₐ. In embodiments, R₅' is C(═O)OH. In embodiments, R₅' is —CH₂ORₐ. In embodiments, R₅' is —CH₂OH. In embodiments, R₅' is —CH₂SRₐ. In embodiments, R₅' is —CH₂SH. In embodiments, R₅' is —CH₂NRₐRₐ. In embodiments, R₅' is —CH₂NH₂. In embodiments, R₅' is —CH₂NHCH₃. In embodiments, R₅' is aryl. In embodiments, R₅' is heteroaryl. In embodiments, R₅' is cycloalkyl. In embodiments, R₅ and R₅' are independently hydrogen, —ORₐ, or fluoro. In embodiments, R₅ and R₅' are independently hydrogen, or —OH. In embodiments, R₅ and R₅' are fluoro.

In embodiments of the compounds of Formula (I), (Ia), (II), (IIa), or (III), R₆ is hydrogen. In embodiments, R₆ is deuterium. In embodiments, R₆ is optionally substituted alkyl. In embodiments, R₆ is CH₃. In embodiments, R₆ is CH₂CH₃. In embodiments, R₆ is optionally substituted aryl. In embodiments, R₆ is optionally substituted heteroalkyl. In embodiments, R₆ is —ORₐ. In embodiments, R₆ is —OH. In embodiments, R is —OCH₃. In embodiments, R₆ is —OCH₂CH₃. In embodiments, R₆ is -alkylene-ORₐ. In embodiments, R₆ is —C(═O)ORₐ. In embodiments, R₆ is —C(═O)NRₐRₐ.

In embodiments of the compounds of Formula (I), (Ia), (II), (IIa), or (III), R₇ is hydrogen. In embodiments, R₇ is deuterium. In embodiments, R₇ is optionally substituted alkyl. In embodiments, R₇ is CH₃. In embodiments, R₇ is CH₂CH₃. In embodiments, R₇ is deuterated alkyl. In embodiments, R₇ is halogenated alkyl. In embodiments, R₇ is fluoro. In embodiments, R₇ is —ORₐ. In embodiments, R₇ is —OH. In embodiments, R₇ is —OCH₃. In embodiments, R is —OCH₂CH₃. In embodiments, R₇ is C(═O)ORₐ. In embodiments, R₇ is C(═O)OH. In embodiments, R₇ is —CH₂ORₐ. In embodiments, R₇ is —CH₂OH. In embodiments, R₇ is —CH₂SRₐ. In embodiments, R₇ is —CH₂SH. In embodiments, R₇ is —CH₂NRₐRₐ. In embodiments, R₇ is —CH₂NH₂. In embodiments, R₇ is —CH₂NHCH₃. In embodiments, R₇ is aryl. In embodiments, R is heteroaryl. In embodiments, R₇ is cycloalkyl. In embodiments, R₇ is optionally substituted alkyl or —CH₂ORₐ.

In embodiments of the compounds of Formula (I), (II), (IIa), or (III), R₉ is hydrogen. In embodiments, R₉ is deuterium. In embodiments, R₉ is optionally substituted alkyl. In embodiments, R₉ is CH₃. In embodiments, R₉ is CH₂CH₃. In embodiments, R₉ is deuterated alkyl. In embodiments, R₉ is halogenated alkyl. In embodiments, R₉ is fluoro. In embodiments, R₉ is —ORₐ. In embodiments, R₉ is —OH. In embodiments, R₉ is —OCH₃. In embodiments, R₉ is —OCH₂CH₃. In embodiments, R₉ is C(═O)ORₐ. In embodiments, R₉ is C(═O)OH. In embodiments, R₉ is —CH₂ORₐ. In embodiments, R₉ is —CH₂OH. In embodiments, R₉ is —CH₂SRₐ. In embodiments, R₉ is —CH₂SH. In embodiments, R₉ is —CH₂NRₐRₐ. In embodiments, R₉ is —CH₂NH₂. In embodiments, R₉ is —CH₂NHCH₃. In embodiments, R₉ is aryl. In embodiments, R₉ is heteroaryl. In embodiments, R₉ is cycloalkyl.

In embodiments of the compounds of Formula (I), (II), (IIa), or (III), R₉′ is hydrogen. In embodiments, R₉′ is deuterium. In embodiments, R₉′ is optionally substituted alkyl. In embodiments, R₉′ is CH₃. In embodiments, R₉′ is CH₂CH₃. In embodiments, R₉′ is deuterated alkyl. In embodiments, R₉′ is halogenated alkyl. In embodiments, R₉′ is fluoro. In embodiments, R₉′ is —ORₐ. In embodiments, R₉′ is —OH. In embodiments, R₉′ is —OCH₃. In embodiments, R₉′ is —OCH₂CH₃. In embodiments, R₉′ is C(═O)ORₐ. In embodiments, R₉′ is —C(═O)OH. In embodiments, R₉′ is —CH₂ORₐ. In embodiments, R₉′ is —CH₂OH. In embodiments, R₉′ is —CH₂SRₐ. In embodiments, R₉′ is —CH₂SH. In embodiments, R₉′ is —CH₂NRₐRₐ. In embodiments, R₉′ is —CH₂N—H₂. In embodiments, R₉′ is —CH₂NHCH₃. In embodiments, R₉′ is aryl. In embodiments, R₉′ is heteroaryl. In embodiments, R₉′ is cycloalkyl.

In embodiments of the compounds of Formula (II), (IIa), or (III), R₉″ is hydrogen. In embodiments, R₉″ is deuterium. In embodiments, R₉″ is optionally substituted alkyl. In embodiments, R₉″ is CH₃. In embodiments, R₉″ is CH₂CH₃. In embodiments, R₉″ is deuterated alkyl. In embodiments, R₉″ is halogenated alkyl. In embodiments, R₉″ is fluoro. In embodiments, R₉″ is —ORₐ. In embodiments, R₉″ is —OH. In embodiments, R₉″ is —OCH₃. In embodiments, R₉″ is —OCH₂CH₃. In embodiments, R₉″ is C(═O)ORₐ. In embodiments, R₉″ is —C(═O)OH. In embodiments, R₉″ is —CH₂ORₐ. In embodiments, R₉″ is —CH₂OH. In embodiments, R₉″ is —CH₂SRₐ. In embodiments, R₉″ is —CH₂SH. In embodiments, R₉″ is —CH₂NRₐRₐ. In embodiments, R₉″ is —CH₂NH₂. In embodiments, R₉″ is —CH₂NHCH₃. In embodiments, R₉″ is aryl. In embodiments, R₉″ is heteroaryl. In embodiments, R₉″ is cycloalkyl.

### Compositions

The present disclosure provides pharmaceutical compositions for treating various conditions or disorders in a patient in need thereof. In some embodiments, a pharmaceutical composition comprises one or more compounds of the present disclosure (e.g., a compound of Formula (I), (Ia), (II), (IIa), (III)), or pharmaceutically acceptable salt, prodrug, or stereoisomer thereof. In embodiments, the pharmaceutical compositions comprise pharmaceutically acceptable excipients and adjuvants.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Methods of Treatment

In one aspect, the present disclosure provides methods of treating a disease or disorder in a patient in need thereof, the methods comprising administering a therapeutically effective amount of a compound described herein (e.g., a compound of Formula (I), (Ia), (II), (IIa), (III)), or pharmaceutically acceptable salt, prodrug, or stereoisomer thereof to the patient.

In embodiments, the present disclosure provides methods of treating alcoholism, substance abuse disorder, or opioid use disorder. In embodiments, the present disclosure provides methods of treating opioid use disorder. In embodiments, the present disclosure provides methods of treating the symptoms of detoxification and/or withdrawal that result from stopping or reducing the use of a medication or drug. In embodiments, the medication or drug is a substance with a high potential for dependency or abuse. In embodiments, the present disclosure provides methods of treating a condition related to compulsive/repetitive behaviors, underlying neurocircuitries and neuroplastic effects (e.g., addictions such as gambling or sex, eating disorders, obsessive compulsive disorder (OCD), major depressive disorder (MDD), treatment-resistant depression (TRD), anxiety, post-traumatic stress disorder)(PTSD), attention-deficit/hyperactivity disorder (ADHD), autism spectrum disorder (ASD), and the like).

In embodiments, the present disclosure provides methods of treating substance abuse disorder in a patient in need thereof, comprising administering a therapeutically effective amount of a compound disclosed herein (e.g., a compound of Formula (I), (Ia), (II), (IIa), (III)), or pharmaceutically acceptable salt, prodrug, or stereoisomer thereof, or a pharmaceutical composition thereof to the patient.

In embodiments, the present disclosure provides methods of treating opioid use disorder in a patient in need thereof, comprising administering a therapeutically effective amount of a compound disclosed herein (e.g., a compound of Formula (I), (Ia), (II), (IIa), (III)), or pharmaceutically acceptable salt, prodrug, or stereoisomer thereof, or a pharmaceutical composition thereof to the patient.

### EXAMPLES

The disclosure now being generally described, it will be more readily understood by reference to the following examples which are included merely for purposes of illustration of certain aspects and embodiments of the present disclosure and are not intended to limit the disclosure.

The compounds of the present disclosure can be synthesized using the methods as hereinafter described below, together with synthetic methods known in the art of synthetic organic chemistry or variations thereon as appreciated by those skilled in the art.

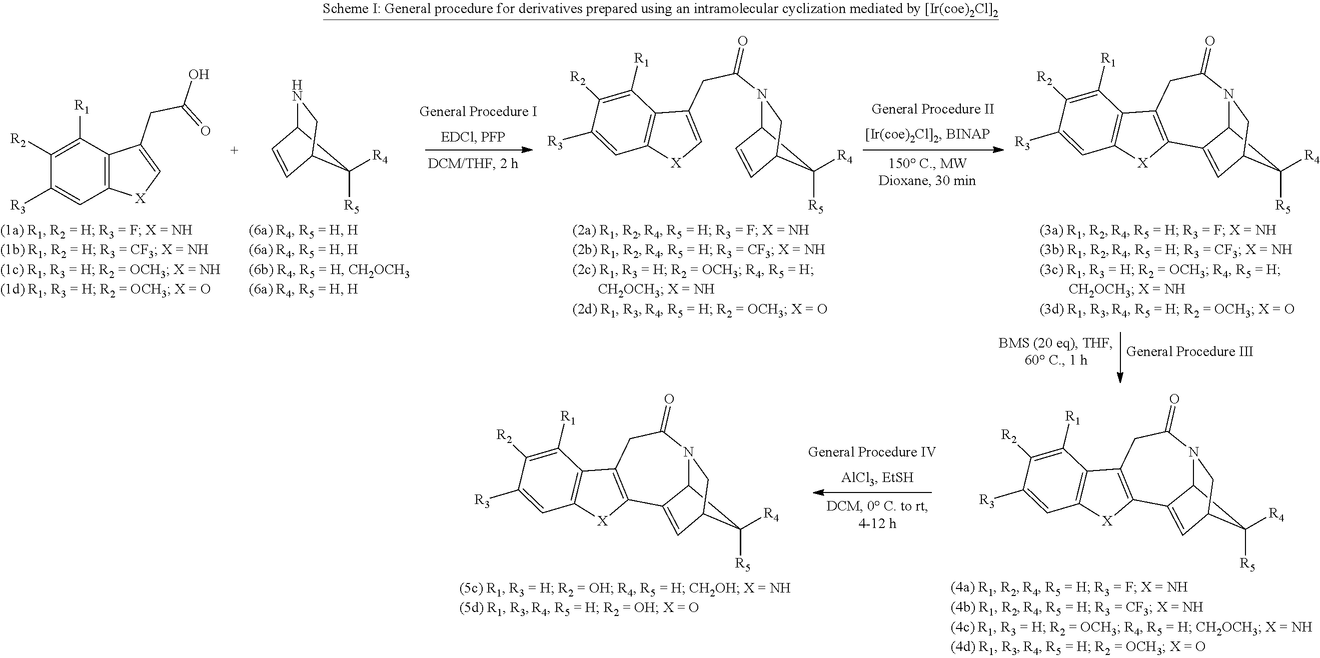

General Procedure (I). Synthesis of [Ir (Coe)₂Cl]₂ Mediated Intramolecular Cyclization Precursor (2)

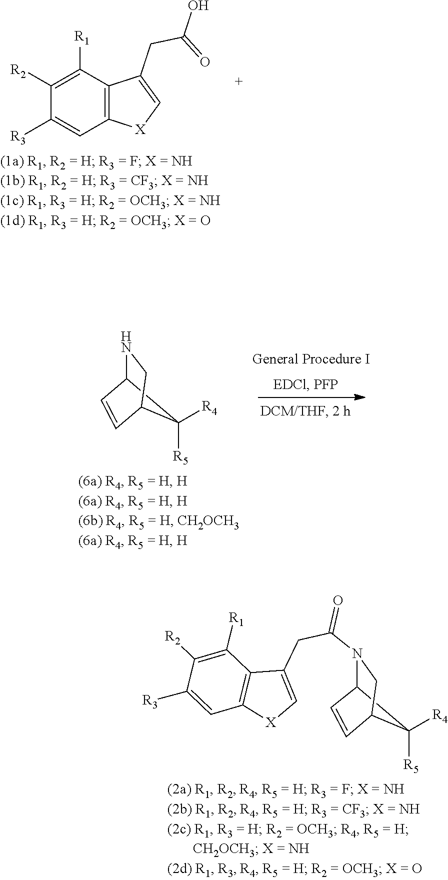

Under an inert atmosphere of argon gas, substituted heteroaryl acetic acid (1)(1 eq) and pentafluoro phenol (1.1 eq) were dissolved in 1:1 of DCM/THF (0.24 M), then EDCI (1.1 eq) was added. The reaction was stirred for 1-2 hrs at rt. After complete consumption of acid added 2-Azabicyclo[2.2.1]hept-5-ene (6a)(6 eq.) to the reaction mixture and continued to stir it for an additional 2 hrs. Removed all the volatiles from the reaction mixture on the rotavapor, redissolved in DCM and washed with water, sodium bicarbonate solution, and brine. Dried the organic layer on sodium sulfate, filter it and the filtrate was concentrated. The crude was purified by normal phase silica gel column chromatography, running a mobile phase of 80-100% Ethyl acetate in Hexane, and the product containing fractions were dried under reduced pressure to afford the desired product (2).

### General Procedure (II). Iridium-Catalyzed Intramolecular Hydroheteroarylation of Bicycloalkene (3)

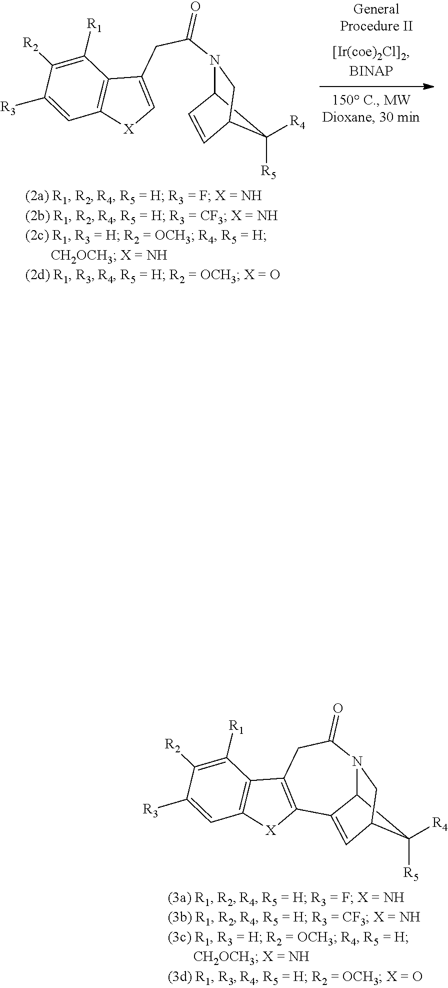

An oven-dried microwave vial was charged with 4 A° molecular sieves (100 mg/2 mL), BINAP(0.3 eq.), the corresponding starting material (2)(1.0 eq.), and a magnetic stir bar. Dioxane (0.36 M) was added to the solids, while purging the argon gas into the reaction mixture added [Ir (coe)₂C1]2 (0.15 eq.) and continued purging the inert gas for an additional 3 minutes. Closed the reaction vial and heated to 150° C. for 30 minutes under the microwave. After bringing it to room temperature reaction mixture was diluted with DCM and passed through celite pad. Filtrate was concentrated and the residue was purified by flash column chromatography to obtain desired product (3).

### General Procedure (III). Amide Reduction of Intramolecular Cyclization Product (4)

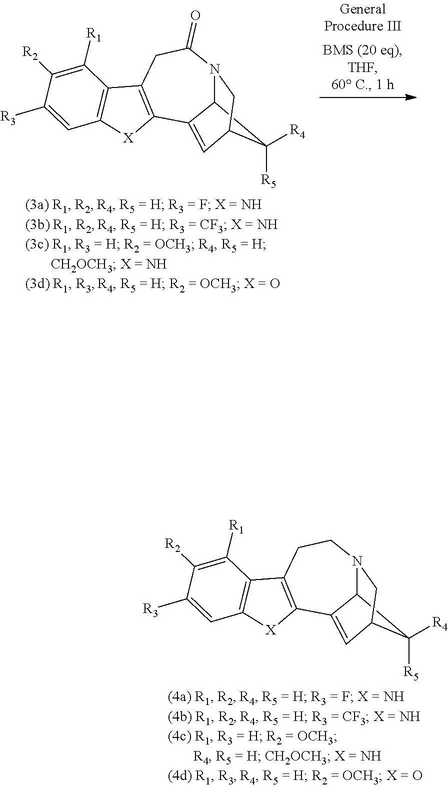

Under an inert atmosphere of argon gas, amide (3)(1 eq) was suspended in THF (0.046 M) and cooled to 0° C., then added borane dimethylsulfide. The reaction was stirred for 30 minutes in an ice bath, removed from the ice bath, and stirred for 1 h at 50° C. After completion of the reaction, it was cooled to 0° C. and quenched with methanol. THF was removed under reduced pressure and added methanol (50 ml), HCl (10 eq. 12M) and stirred at 60° C. for 2 hrs. Then the volatiles were removed under reduced pressure, and the crude reaction mixture was purified by reverse phase silica gel column chromatography and the product-containing fractions were dried under reduced pressure to obtain desired product (4).

### General Procedure (IV); Synthesis of Noribogalogs (5)

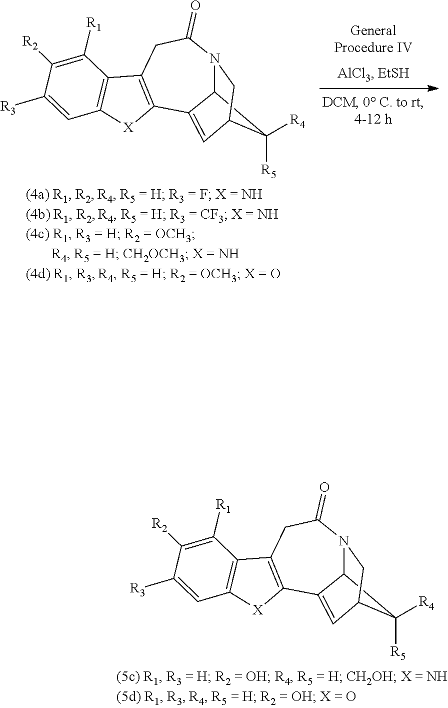

Under an inert atmosphere of argon gas, corresponding ibogalog derivative (4)(1 eq.) was dissolved in DCM (0.016 M) and cooled to 0° C. While stirring at 0° C., aluminum chloride (6 eq) and ethanethiol (12 eq) were added in one portion and caped. The mixture was moved to rt and stirred for 1 h at rt. Then it was cooled to 0° C. and quenched with dropwise addition of sodium hydroxide solution (18 eq). The pH should be between 7 to 8. Then 4 mL of methanol was added to facile stirring and stirred for 5 minutes. The solvents were removed by reduced pressure and placed under a high vacuum for 1 h. The crude reaction mixture was purified by normal phase silica gel column chromatography, running a mobile phase of 20% MeOH in 80% DCM, and the product-containing fractions were dried under reduced pressure to afford the desired product (5).

### Example 1:1-((1S,4R)-2-Azabicyclo[2.2.1]Hept-5-En-2-Yl)-2-(6-Fluoro-1H-Indol-3-Yl) Ethan-1-One (2a)

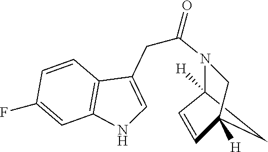

The title compound 1-((1S,4R)-2-azabicyclo[2.2.1]hept-5-en-2-yl)-2-(6-fluoro-1H-indol-3-yl) ethan-1-one (2a), was prepared according to the protocol described in general procedure (I) to afford the desired product (220 mg). ¹H NMR (400 MHZ, MeOD)δ 7.58-7.46 (m, 1H), 7.20-6.99 (m, 2H), 6.86-6.76 (m, 1H), 6.40-6.27 (m, 1H), 5.91-5.85 (m, 0.5H), 5.11-5.06 (m, 0.5H), 4.89 (s, 1H), 3.94-3.78 (m, 1.2H), 3.71-3.57 (m, 1.3H), 3.42-3.36 (m, 0.5H), 3.31-3.20 (m, 1H), 2.89-2.72 (m, 1H), 1.68-1.52 (m, 2H). ESI-MS: measured m/z 271.13 [M+1]⁺.

### Example 2:1-((1S,4R)-2-Azabicyclo[2.2.1]Hept-5-En-2-Yl)-2-(6-(Trifluoromethyl)-1H-Indol-3-Yl) Ethan-1-One (2b)

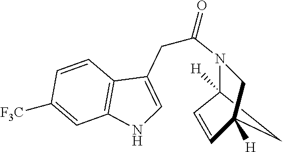

The title compound 1-((1S,4R)-2-azabicyclo[2.2.1]hept-5-en-2-yl)-2-(6-(trifluoromethyl)-1H-indol-3-yl) ethan-1-one (2b), was prepared according to the protocol described in general procedure (I) to afford the desired product 460 mg, ¹H NMR (400 MHZ, CDCl₃)δ 8.55 (s, 1H), 7.76-7.64 (m, 1H), 7.62 (d, J=8.6 Hz, 1H), 7.36 (td, J=8.4, 1.6 Hz, 1H), 7.23 (dd, J=12.0, 2.4 Hz, 1H), 6.47-6.41 (m, 0.5H), 6.35-6.29 (m, 1H), 6.04-5.98 (m, 0.5H), 5.24-5.20 (m, 0.5H), 4.73-4.69 (m, 0.5H), 3.86 (d, J=3.2 Hz, 1H), 3.68 (dd, J=7.2, 1.0 Hz, 1H), 3.59-3.43 (m, 1H), 3.33-3.22 (m, 1H), 2.92-2.79 (m, 1H), 1.69-1.64 (m, 2H). ESI-MS: measured m/z 321.07 [M+1]⁺.

### Example 3: Synthesis of (+)-2-(4-Methoxybenzyl)-7-(Methoxymethyl)-2-Azabicyclo[2.2.19 Hept-5-En-3-One (6b)

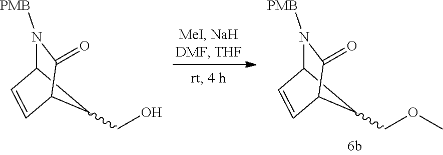

(+)-7-(hydroxymethyl)-2-(4-methoxybenzyl)-2-azabicyclo[2.2.1]hept-5-en-3-one ((1100 mg, 506.85 mmol) was dissolved in DMF (4 mL) and THF (4 mL) and cooled to 0° C. Then Sodium hydride (203.63 mg, 5.09 mmol, 60% purity) was added followed by Iodomethane (842.98 mg, 5.94 mmol, 369.73 μL). The mixture was warmed to room temperature and stirred for 4 h. Water (100 mL) was added to the reaction mixture and stirring it for 2 min, crude product was extracted with ethyl acetate (2×75 mL). The residue was purified by flash chromatography on silica gel using MeOH and DCM as a mobile phase to give the desired product (6b)(900 mg). ESI-MS: measured m/z 274.1 [M+H]⁺.

### Example 4:2-(5-Methoxy-1H-Indol-3-Yl)-1-((1R,4S)-7-(Methoxymethyl)-2-Azabicyclo[2.2.1]Hept-5-En-2-Yl) Ethan-1-One (2c)

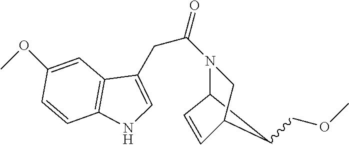

The title compound 2-(5-methoxy-1H-indol-3-yl)-1-((1R,4S)-7-(methoxymethyl)-2-azabicyclo[2.2.1]hept-5-en-2-yl) ethan-1-one (2c), was prepared according to the protocol described in general procedure (I), starting from 5-Methoxy-1H-indol-3-acetic acid (1c) and (+)-2-(4-methoxybenzyl)-7-(methoxymethyl)-2-azabicyclo[2.2.1]hept-5-en-3-one (6b), to afford the desired product (300 mg). ¹H NMR (400 MHZ, CDCl₃)δ 8.24 (s, 1H), 7.22-7.10 (m, 2H), 7.20-6.99 (m, 1H), 6.47-6.30 (m, 1.5H), 6.00-5.93 (m, 0.5H), 5.01-5.96 (m, 0.5H), 4.60-4.54 (m, 0.5H), 3.86-3.85 (m, 3H), 3.77-3.74 (m, 1H), 3.37-3.27 (m, 1H), 3.15-3.09 (m, 2H), 3.07-2.98 (m, 2H), 2.87-2.72 (m, 2H), 2.15-2.02 (m, 1H), 1.63 (s, 1H), 1.31-1.21 (m, 1H). ESI-MS: measured m/z 405.91 [M]⁺.

### Example 5: (2S,12R,12aS)-9-Fluoro-2,3,6,11,12,12a-Hexahydro-2,12 Methanopyrrolo[1′,2′: 1,2]Azepino[4,5-b]Indol-5 (1H)-One (3a)

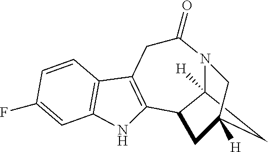

The title compound (2S,12R, 12aS)-9-fluoro-2,3,6,11,12,12a-hexahydro-2,12-methanopyrrolo[1′,2′: 1,2]azepino[4,5-b]indol-5 (1H)-one (3a), was prepared according to the protocol described in general procedure (II), starting from 1-((1S,4R)-2-azabicyclo[2.2.1]hept-5-en-2-yl)-2-(6-fluoro-1H-indol-3-yl) ethan-1-one (2a) to afford the desired product (80 mg). 1H NMR (400 MHZ, MeOD)δ 7.42 (dd, J=8.7, 5.2 Hz, 1H), 6.96 (dd, J=9.8, 2.4 Hz, 1H), 6.80 (ddd, J=9.7, 8.6, 2.3 Hz, 1H), 4.72 (s, 1H), 4.08 (dd, J=14.6, 2.3 Hz, 1H), 3.50 (d, J=14.6 Hz, 1H), 3.44-3.36 (m, 2H), 3.27 (dd, J=10.1, 2.2 Hz, 1H), 2.73-2.69 (m, 1H), 2.46-2.35 (m, 1H), 2.09 (d, J=10.1 Hz, 1H), 1.95 (d, J=10.4 Hz, 1H), 1.36-1.27 (m, 1H). ESI-MS: measured m/z 271.27 [M+1]+

### Example 6: (2S,12R,12aS)-9-(Trifluoromethyl)-2,3,6,11,12,12a-Hexahydro-2,12 Methanopyrrolo[1′,2′: 1,2]Azepino[4,5-b]Indol-5 (1H)-One (3b)

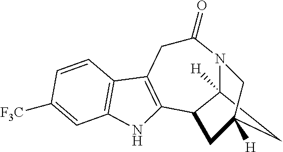

The title compound (2S,12R,12aS)-9-(trifluoromethyl)-2,3,6,11,12,12a-hexahydro-2,12-methanopyrrolo[1′,2′: 1,2]azepino[4,5-b]indol-5 (1H)-one (3b), was prepared according to the protocol described in general procedure (II), starting from 1-((1S,4R)-2-azabicyclo[2.2.1]hept-5-en-2-yl)-2-(6-(trifluoromethyl)-1H-indol-3-yl) ethan-1-one (2b) to afford the desired product (78 mg). ¹H NMR (400 MHZ, MeOD)δ 7.63 (d, J=8.4 Hz, 1H), 7.57-7.55 (m, 1H), 7.28 (dd, J=8.3, 1.6 Hz, 1H), 4.76 (s, 1H), 4.13 (dd, J=14.6, 2.1 Hz, 1H), 3.57 (d, J=14.7 Hz, 1H), 3.51-3.38 (m, 1H), 3.30-3.18 (m, 2H), 2.76-2.70 (m, 1H), 2.55-2.39 (m, 1H), 2.16-1.94 (m, 2H), 1.38-1.31 (m, 1H). ESI-MS: measured m/z 321.27 [M+1]⁺.

### Example 7:8-Methoxy-1-(Methoxymethyl)-2,3,6,11,12,12a-Hexahydro-2,12-Methanopyrrolo[1′,2′: 1,2]Azepino[4,5-b]Indol-5 (1H)-One (3c)

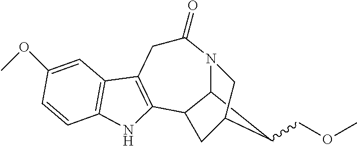

The title compound 8-methoxy-1-(methoxymethyl)-2,3,6,11,12,12a-hexahydro-2,12-methanopyrrolo[1′,2′: 1,2]azepino[4,5-b]indol-5 (1H)-one (3c), was prepared according to the protocol described in general procedure (II), starting from 2-(5-methoxy-1H-indol-3-yl)-1-((1R,4S)-7-(methoxymethyl)-2-azabicyclo[2.2.1]hept-5-en-2-yl) ethan-1-one (2c) to afford the desired product (80 mg). ¹H NMR (400 MHZ, CDCl₃)δ 8.21 (s, 1H), 7.11 (d, J=8.7 Hz, 1H), 6.97 (d, J=2.4 Hz, 1H), 6.77 (dd, J=8.7, 2.4 Hz, 1H), 4.43 (s, 1H), 3.94 (dd, J=14.5, 2.1 Hz, 1H), 3.84 (s, 3H), 3.65-3.46 (m, 4H), 3.42 (s, 3H), 3.39-3.32 (m, 1H), 3.28 (d, J=10.6 Hz, 1H), 2.54-2.47 (m, 1H), 2.39 (t, J=7.4 Hz, 1H), 2.37-2.25 (m, 1H), 1.36 (dd, J=12.6, 5.3 Hz, 1H). ESI-MS: measured m/z 327.27 [M+1]⁺.

### Example 8: (2S,12R,12aS)-9-Fluoro-1,2,3,5,6,11,12,12a-Octahydro-2,12-Methanopyrrolo[1′,2′: 1,2]Azepino[4,5-b]Indole Formic Acid Salt (4a)

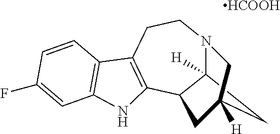

The title compound (2S,12R, 12aS)-9-fluoro-1,2,3,5,6,11,12,12a-octahydro-2,12-methanopyrrolo[1′,2′: 1,2]azepino[4,5-b]indole formic acid salt (4a), was prepared (5.5 mg) according to the protocol described in general procedure (III) starting from (2S,12R,12aS)-9-fluoro-2,3,6,11,12,12a-hexahydro-2,12-methanopyrrolo[1′,2′: 1,2]azepino[4,5-b]indol-5 (1H)-one (3a). ¹H NMR (400 MHZ, MeOD)δ 8.56 (brs, 1H), 7.42 (dd, J=8.7, 5.2 Hz, 1H), 6.99 (dd, J=9.8, 2.3 Hz, 1H), 6.87-6.77 (m, 1H), 4.28-4.21 (m, 1H), 3.72-3.49 (m, 4H), 3.38 (d, J=11.0 Hz, 1H), 3.33-3.11 (m, 3H), 2.90-2.84 (m, 1H), 2.57-2.44 (m, 1H), 2.14-2.03 (m, 2H), 1.60-1.52 (m, 1H). ¹³C NMR (101 MHz, MeOD) δ 118.3 (CH), 118.2 (CH), 107.2 (CH), 106.9 (CH), 96.5 (CH), 96.3 (CH), 65.6 (CH), 53.5 (CH₂), 50.5 (CH₂), 38.4 (CH), 38.3 (CH₂), 36.6 (CH), 35.7 (CH₂), 17.2 (CH₂). ESI-MS: measured m/z 257.27 [M+H]⁺. Purity by HPLC: 98.6% at 254 nm.

### Example 9: (2S,12R,12aS)-9-(Trifluoromethyl)-1,2,3,5,6,11,12,12a-Octahydro-2,12-Methanopyrrolo[1′,2′: 1,2]Azepino[4,5-b]Indole Formic Acid Salt (4b)

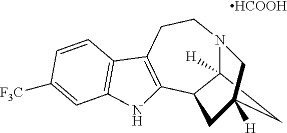

The title compound (2S,12R, 12aS)-9-(trifluoromethyl)-1,2,3,5,6,11,12,12a-octahydro-2,12-methanopyrrolo[1′,2′: 1,2]azepino[4,5-b]indole formic acid salt (4b), was prepared (9.8 mg, 11% yield) according to the protocol described in general procedure (III) starting from (2S,12R, 12aS)-9-(trifluoromethyl)-2,3,6,11,12,12a-hexahydro-2,12-methanopyrrolo[1′,2′: 1,2]azepino[4,5-b]indol-5 (1H)-one (3b). ¹H NMR (400 MHZ, MeOD)δ 7.59 (d, J=8.4 Hz, 1H), 7.57 (s, 1H), 7.27 (dd, J=8.4, 1.6 Hz, 1H), 4.00-3.92 (m, 1H), 3.58-3.45 (m, 4H), 3.31 (s, 1H), 3.22-3.17 (m, 1H), 3.05-2.98 (m, 1H), 2.80-2.76 (m, 1H), 2.51-2.41 (m, 1H), 2.00-1.92 (m, 2H), 1.55-1.48 (m, 1H). ¹H NMR (376 MHz, MeOD)8-61.8. 13C NMR (101 MHZ, MeOD)δ 117.5 (CH), 115.0 (CH), 114.9 (CH), 107.6 (CH), 107.5 (CH), 64.5 (CH), 52.7 (CH₂), 49.8 (CH₂), 40.8 (CH), 38.7 (CH₂), 37.3 (CH), 36.6 (CH₂). 17.7 (CH₂). ESI-MS: measured m/z 307.27 [M+H]⁺. Purity by HPLC: 98.6% at 254 nm.

### Example 10: (8-Methoxy-1-(Methoxymethyl)-1,2,3,5,6,11,12,12a-Octahydro-2,12 Methanopyrrolo[1′,2′: 1,2]Azepino[4,5-b]Indole Formic Acid Salt (4c)

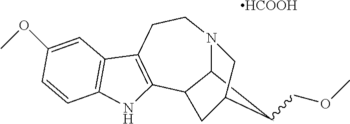

The title compound (8-methoxy-1-(methoxymethyl)-1,2,3,5,6,11,12,12a-octahydro-2,12-methanopyrrolo[1′,2′: 1,2]azepino[4,5-b]indole formic acid salt (4c), was prepared (13.0 mg) according to the protocol described in general procedure (III) starting from 8-methoxy-1-(methoxymethyl)-2,3,6,11,12,12a-hexahydro-2,12-methanopyrrolo[1′,2′: 1,2]azepino[4,5-b]indol-5 (1H)-one (3c). ¹H NMR (400 MHz, MeOD) δ 7.16 (d, J=8.7 Hz, 1H), 6.95 (d, J=2.4 Hz, 1H), 6.74 (dd, J=8.7, 2.4 Hz, 1H), 4.04-3.99 (m, 1H), 3.82 (s, 3H), 3.73-3.64 (m, 2H), 3.63-3.57 (m, 2H), 3.56-3.49 (m, 2H), 3.43 (s, 3H), 3.32-3.19 (m, 2H), 3.08-2.98 (m, 1H), 2.68-2.62 (m, 1H), 2.60-2.46 (m, 2H), 1.54-1.46 (m, 1H). ¹³C NMR (101 MHZ, MeOD)δ 111.0 (CH), 110.9 (CH), 99.2 (CH), 68.7 (CH₂), 66.2 (CH), 58.0 (CH), 54.9 (CH₃), 52.2 (CH₂), 51.3 (CH₂), 50.7 (CH₃), 40.1 (CH), 38.5 (CH), 36.9 (CH₂), 17.9 (CH₂). ESI-MS: measured m/z 313.33 [M+H]⁺. Purity by HPLC: 98.5% at 254 nm.

### Example 11:1-(Hydroxymethyl)-1,2,3,5,6,11,12,12a-Octahydro-2,12 Methanopyrrolo[1′,2′: 1,2]Azepino[4,5-b]Indol-8-Ol Formic Acid Salt (5c)

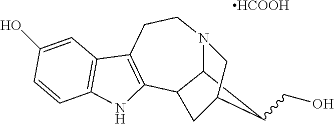 [SMILES: "·HCOOH" ; OCC1C2CC3c4[nH]c5ccc(O)cc5c4CCN(C2)C31]

The title compound 1-(hydroxymethyl)-1,2,3,5,6,11,12,12a-octahydro-2,12 methanopyrrolo[1′,2′: 1,2]azepino[4,5-b]indol-8-ol formic acid salt (5c), was prepared (7.5 mg) according to the protocol described in general procedure (IV) starting from (8-methoxy-1-(methoxymethyl)-1,2,3,5,6,11,12,12a-octahydro-2,12-methanopyrrolo[1′,2′: 1,2]azepino[4,5-b]indole formic acid salt (4c). ¹H NMR (400 MHZ, MeOD)δ 7.11 (d, J=8.6 Hz, 1H), 6.83 (d, J=2.3 Hz, 1H), 6.65 (dd, J=8.6, 2.3 Hz, 1H), 4.12-4.06 (m, 1H), 3.95-3.83 (m, 2H), 3.71-3.61 (m, 2H), 3.61-3.50 (m, 2H), 3.28-3.16 (m, 1H), 3.05-2.96 (m, 1H), 2.71-2.66 (m, 1H), 2.58-2.48 (m, 2H), 1.57-1.47 (m, 1H). ¹³C NMR (101 MHZ, MeOD) § 110.8 (CH), 110.8 (CH), 101.3 (CH), 66.6 (CH), 58.0 (CH₂), 52.5 (CH), 52.3 (CH₂), 51.3 (CH₂), 39.7 (CH), 38.4 (CH), 36.9 (CH₂), 17.8 (CH₂). ESI-MS: measured m/z 285.27 [M+H]⁺. Purity by HPLC: 95.4% at 254 nmD

### Example 12: Synthesis of ((6S,7S,9S,11S)-7-Ethyl-2-Methoxy-7,8,9,10,12,13-Hexahydro-5H-6,9-Methanopyrido[1′,2′: 1,2]Azepino[4,5-b]Indol-6 (6aH)-Yl) Methanol Formic Acid Salt (8)

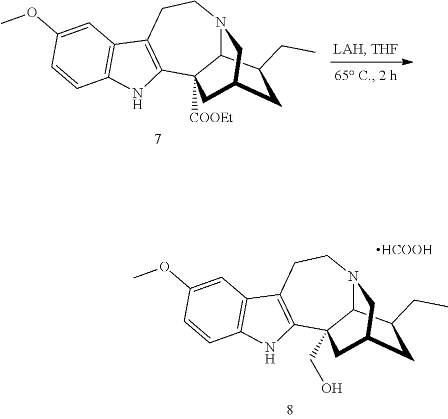

Lithium aluminium hydride (25.75 mg, 678.48 μmol) was transferred to a 1-dram vial under an inert atmosphere, 1 mL of THF was added. Then solution of methyl 18-ethyl-12-methoxy-22,23-diazapentacyclononadeca-3,5 (12), 13,15-tetraene-21-carboxylate (7)(50 mg, 135.70 μmol) in 3 ml THF was added slowly to the reaction mixture and stirred for 1 min and reaction vial was sealed under argon and refluxed for 2 h. After cooling the reaction mixture to 0° C., quenched with water and extracted with EtOAc. The organic layer was dried on sodium sulfate, and filtered. Filtrate was concentrated and the residue was purified on the reverse phase column. Isolated the desired compound (8)(18.0 mg). ¹H NMR (400 MHZ, MeOD)δ 7.19 (d, J=8.7 Hz, 1H), 6.94 (d, J=2.4 Hz, 1H), 6.72 (dd, J=8.7, 2.4 Hz, 1H), 4.01-3.94 (m, 1H), 3.83 (s, 3H), 3.79-3.72 (m, 1H), 3.67-3.55 (m, 1H), 3.26-2.97 (m, 4H), 2.96-2.88 (m, 1H), 2.19-1.98 (m, 2H), 1.87 (d, J=17.9 Hz, 2H), 1.70-1.56 (m, 2H), 1.50-1.42 (m, 1H), 1.26-1.18 (m, 1H), 1.02 (t, J=7.4 Hz, 3H). ESI-MS: measured m/z 341.33 [M+H]⁺. Purity by HPLC: 98.9% at 254 nm.

### Example 13: (6S,7S,9S,11S)-7-Ethyl-6-(Hydroxymethyl)-6,6a,7,8,9,10,12,13-Octahydro-5H-6,9-Methanopyrido[1′,2′: 1,2]Azepino[4,5-b]Indol-2-Ol (9)

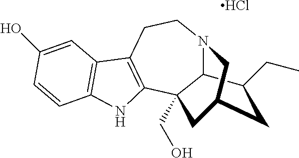

The title compound (6S,7S,9S,11S)-7-ethyl-6-(hydroxymethyl)-6,6a, 7,8,9,10,12,13-octahydro-5H-6,9-methanopyrido[1′,2′: 1,2]azepino[4,5-b]indol-2-ol (9), was prepared (6.5 mg) according to the protocol described in general procedure (IV) starting from ((6S,7S,9S,11S)-7-ethyl-2-methoxy-7,8,9,10,12,13-hexahydro-5H-6,9-methanopyrido[1′,2′: 1,2]azepino[4,5-b]indol-6 (6aH)-yl) methanol (8). ¹H NMR (400 MHZ, MeOD)δ 7.18 (d, J=8.6 Hz, 1H), 6.88-6.82 (m, 1H), 6.69 (dd, J=8.6, 2.3 Hz, 1H), 4.35-4.26 (m, 1H), 4.06-3.94 (m, 2H), 3.90-3.82 (m, 1H), 3.52-3.41 (m, 2H), 3.32-3.26 (m, 1H), 3.17-3.04 (m, 2H), 2.44-2.31 (m, 1H), 2.29-2.20 (m, 1H), 2.18-2.03 (m, 2H), 1.83-1.68 (m, 2H), 1.65-1.57 (m, 1H), 1.44-1.36 (m, 1H), 1.09 (t, J=7.3 Hz, 3H). ¹³C NMR (101 MHZ, MeOD)δ 111.4 (CH), 111.0 (CH), 101.5 (CH), 66.7 (CH₂), 57.2 (CH), 56.8 (CH₂), 56.0 (CH₂), 34.0 (CH₂), 32.8 (CH), 28.7 (CH₂), 25.6 (CH), 18.0 (CH₂), 10.5 (CH₃). ESI-MS: measured m/z 327.27 [M+H]⁺. Purity by HPLC: 98% at 254 nm.

### Example 14: Synthesis of (2S,12S,12aS)-8-Methoxy-1,2,3,6,11,12a-Hexahydro-2,12-Methanopyrrolo[1′,2′: 1,2]Azepino[4,5-b]Indole-12 (5H)-Carbonitrile (11)

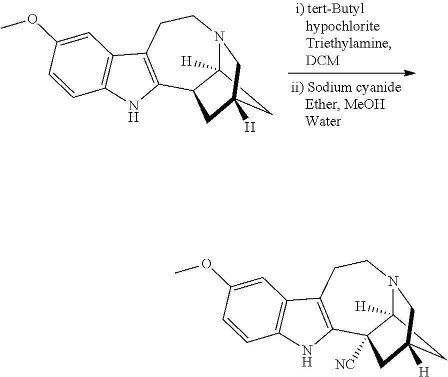

To a stirred solution of (2S,12R, 12aS)-8-methoxy-1,2,3,5,6,11,12,12a-octahydro-2,12-methanopyrrolo[1′,2′: 1,2]azepino[4,5-b]indole (50 mg, 164.03 μmol)(10) in DCM (2 mL) at 0° C. in an ice bath Triethylamine (33.20 mg, 328.07 μmol, 45.73 μL) was added. Next, tert-Butyl hypochlorite (21.37 mg, 196.84 μmol) was diluted in carbon tetrachloride (550 μL) added to the reaction mixture at the same temperature for over 20 minutes. After the addition, the reaction continued for 40 minutes. The reaction mixture was treated with cold water and diluted with methylene chloride and the organic phase was successively washed with water, dried on sodium sulfate, filtered. Filtrate was concentrated under 20° C., crude reaction mixture was taken to the next step without further purification. Crude reaction mixture was treated with sodium cyanide (127.18 mg, 2.54 mmol) was in Methanol (1.47 mL), Water (147.00 μL) and Ether (294.00 μL) in a vial at room temperature for 72 hrs. The reaction mixture was treated with sodium carbonate solution and diluted with DCM and the organic phase was successively washed with water and dried over sodium sulfate and evaporated. The residue was purified by normal phase column chromatography running a mobile phase of 0-10% MeOH in DCM, and the product containing fractions were dried under reduced pressure to afford the desired product (11)(4.1 mg). ¹H NMR (400 MHZ, MeOD)δ 8.30 (brs, 1H), 7.25 (d, J=8.7 Hz, 1H), 6.94 (d, J=2.4 Hz, 1H), 6.78 (dd, J=8.8, 2.4 Hz, 1H), 4.07-4.03 (m, 1H), 3.83 (s, 3H), 3.53-3.41 (m, 1H), 3.25-3.09 (m, 3H), 2.88-2.79 (m, 1H), 2.74-2.68 (m, 2H), 2.68-2.59 (m, 2H), 2.17-2.10 (m, 1H), 2.07-1.98 (m, 1H), 1.90-1.82 (m, 1H). ¹³C NMR (101 MHZ, MeOD)δ 111.8 (CH), 111.3 (CH), 99.6 (CH), 67.7 (CH), 54.8 (CH₃), 52.7 (CH₂), 49.0 (CH₂), 46.1 (CH₂), 39.0 (CH₂), 38.0 (CH), 20.3 (CH₂). ESI-MS: measured m/z 294.27 [M+H]⁺. Purity by HPLC: 97.8% at 254 nm.

### Example 15: (2S,12S,12aS)-8-Hydroxy-1,2,3,6,11,12a-Hexahydro-2,12-Methanopyrrolo[1′,2′: 1,2]Azepino[4,5-b]Indole-12 (5H)-Carbonitrile (12)

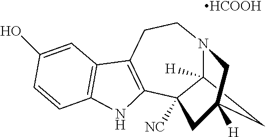

The title compound (2S,12S, 12aS)-8-hydroxy-1,2,3,6,11,12a-hexahydro-2,12-methanopyrrolo[1′,2′: 1,2]azepino[4,5-b]indole-12 (5H)-carbonitrile (12), was prepared (8.2 mg). according to the protocol described in general procedure (IV) starting from (2S,12S, 12aS)-8-methoxy-1,2,3,6,11,12a-hexahydro-2,12-methanopyrrolo[1′,2′: 1,2]azepino[4,5-b]indole-12 (5H)-carbonitrile (11). ¹H NMR (400 MHZ, MeOD)δ 8.22 (s, 1H), 7.17 (d, J=8.6 Hz, 1H), 6.80 (d, J=2.3 Hz, 1H), 6.68 (dd, J=8.7, 2.3 Hz, 1H), 4.13-4.07 (m, 1H), 3.54-3.41 (m, 1H), 3.28-3.14 (m, 2H), 3.14-2.99 (m, 1H), 2.85-2.74 (m, 1H), 2.74-2.58 (m, 3H), 2.18-2.11 (m, 1H), 2.06-1.98 (m, 1H), 1.88-1.77 (m, 1H). ¹³C NMR (101 MHZ, MeOD)δ 111.8 (CH), 111.2 (CH), 108.2 (CH), 67.6 (CH), 52.9 (CH₂), 49.4 (CH₂), 45.8 (CH₂), 38.6 (CH₂), 37.7 (CH), 20.0 (CH₂). ESI-MS: measured m/z 280.27 [M+H]⁺. Purity by HPLC: 94.7% at 254 nm.

### Example 16: (2S,12R,12aS)-8-Methoxy-1,2,3,5,6,12a-Hexahydro-12H-2,12-Methanobenzofuro[2,3-d]Pyrrolo[1,2-a]Azepine-12-Carbonitrile (13)

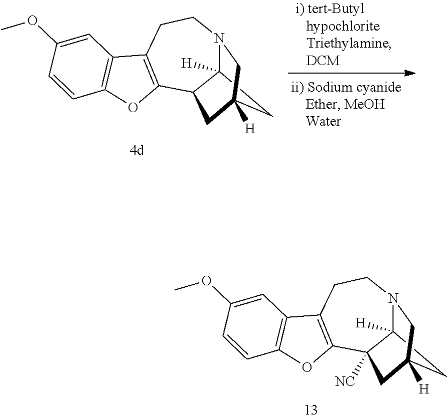

The title compound (2S,12R,12aS)-8-methoxy-1,2,3,5,6,12a-hexahydro-12H-2,12-methanobenzofuro[2,3-d]pyrrolo[1,2-a]azepine-12-carbonitrile (13), was prepared (3.5 mg, 6.6% yield) following the synthetic procedure used for compound 11, starting from (2S,12R,12aS)-6a-chloro-8-methoxy-1,2,3,5,6,6a, 12,12a-octahydro-2,12-methanobenzofuro[2,3-d]pyrrolo[1,2-aJazepin-11-ium (4d) following the synthetic procedure compound. ¹H NMR (400 MHZ, MeOD) δ 7.21 (d, J=8.8 Hz, 1H), 6.95 (d, J=2.6 Hz, 1H), 6.80 (dd, J=8.9, 2.6 Hz, 1H), 3.89-3.85 (m, 1H), 3.83 (s, 3H), 3.66-3.62 (m, 1H), 3.48-3.42 (m, 1H), 3.42-3.39 (m, 1H), 3.39-3.36 (m, 1H), 3.14-3.01 (m, 1H), 2.91-2.86 (m, 1H), 2.62-2.52 (m, 1H), 2.44-2.32 (m, 1H), 2.04-1.96 (m, 1H), 1.75-1.68 (m, 1H), 1.45-1.39 (m, 1H), 0.96-0.88 (m, 1H). ESI-MS: measured m/z 295.20 [M+H]⁺. Purity by HPLC: 98.6% at 254 nm.

### Example 17:1-((1S,4R)-2-Azabicyclo[2.2.1]Hept-5-En-2-Yl)-2-(5-Methoxybenzofuran-3-Yl) Ethan-1-One (2d)

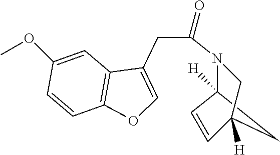 [SMILES: COc1ccc2occ(CC(=O)N3C[C@H]4C=C[C@@H]3C4)c2c1]

The title compound 1-((1S,4R)-2-azabicyclo[2.2.1]hept-5-en-2-yl)-2-(5-methoxybenzofuran-3-yl) ethan-1-one (2d), was prepared (6 mg) according to the protocol described in general procedure (I) starting from 2-(5-Methoxybenzofuran-3-yl) acetic acid (1d) and (1S,4R)-2-azabicyclo[2.2.1]hept-2-ene (6a). ¹H NMR (400 MHZ, CDCl₃)δ 7.56 (m, 1H), 7.40-7.32 (m, 1H), 7.07 (m, 1H), 6.91 (m, 1H), 6.43 (m, 0.5H), 6.32 (m, 1H), 6.09 (m, 0.5H), 5.21 (m, 0.5H), 4.69 (m, 0.5H), 3.87 (s, 3H), 3.73 (m, 1H), 3.58-3.43 (m, 2H), 3.27-3.21 (m, 1H), 2.8-2.78 (m, 1H), 1.70-1.56 (m, 2H). ESI-MS: measured m/z 284.1 [M+H]⁺.

### Example 18:1-((1S,4R)-2-Azabicyclo[2.2.1]Hept-5-En-2-Yl)-2-(5-Hydroxybenzofuran-3-Yl) Ethan-1-One (14)

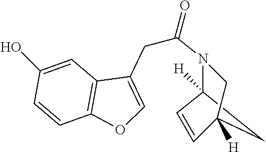 [SMILES: O=C(Cc1coc2ccc(O)cc12)N1C[C@H]2C=C[C@@H]1C2]

The title compound 1-((1S,4R)-2-azabicyclo[2.2.1]hept-5-en-2-yl)-2-(5-hydroxybenzofuran-3-yl) ethan-1-one (14), was prepared (1.4 mg) according to the protocol described in general procedure (IV) starting from 1-((1S,4R)-2-azabicyclo[2.2.1]hept-5-en-2-yl)-2-(5-methoxybenzofuran-3-yl) ethan-1-one (2d). ¹H NMR (400 MHZ, MeOD)δ 7.64-7.59 (m, 1H), 7.28 (d, J=8.8 Hz, 1H), 6.99-6.93 (m, 1H), 6.79 (dd, J=8.8, 2.5 Hz, 1H), 5.89-5.77 (m, 1.5H), 5.74-5.67 (m, 0.5H), 4.81-4.73 (m, 1H), 4.51-4.44 (m, 0.5H), 3.59-3.53 (m, 2H), 3.32-3.29 (m, 0.5H), 3.29-3.23 (m, 1.5H), 2.88-2.77 (m, 0.5H), 2.63-2.54 (m, 0.5H), 2.46 - 2.34 (m, 0.5H), 2.29-2.17 (m, 0.5H), 2.04-1.93 (m, 0.5H), 1.39-1.28 (m, 2H). ESI-MS: measured m/z 270.07 [M+H]⁺. Purity by HPLC: 98.2% at 254 nm.

### Example 19: (1S,4R)-2-(2-(5-Methoxybenzofuran-3-Yl)Ethyl)-2-Azabicyclo[2.2.1]Hept-5-Ene (15)

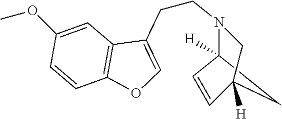 [SMILES: COc1ccc2occ(CCN3C[C@H]4C=C[C@@H]3C4)c2c1]

A 50 mL RBF was charged with Lithium aluminium hydride (60.28 mg, 1.59 mmol) under inert atmosphere, cooled to 0° C., added 3 mL of THF. Then, 1-(5-azabicyclo[2.2.1]hept-2-en-5-yl)-2-(5-methoxybenzofuran-3-yl) ethanone (150 mg, 529.43 μmol)(14) was added slowly to the reaction mixture by dissolving in 2 mL THF at the same temperature. Next, reaction temperature was brought to room temperature and stirred for 10 min. Then reaction left to reflux for an hour. Reaction was monitored by LC-MS. Reaction was quenched by addition of water (1-2 mL) at 0° C. Reaction mixture was diluted with EtOAc and passed it through celite-sodium sulfate pad. Filtrate was concentrated and the residue was purified by normal phase column chromatography running a mobile phase of 0-20% MeOH in DCM, and the product containing fractions were dried under reduced pressure to afford the desired product (15)(111.0 mg). ¹H NMR (400 MHZ, MeOD)δ 7.65 (s, 1H), 7.38 (d, J=8.9 Hz, 1H), 7.13 (d, J=2.6 Hz, 1H), 6.94 (dd, J=9.0, 2.6 Hz, 1H), 6.83-6.72 (m, 1H), 6.37-6.29 (m, 1H), 4.67-4.56 (m, 1H), 3.87 (s, 3H), 3.54-3.46 (m, 1H), 3.19-3.06 (m, 3H), 2.45-2.37 (m, 1H), 1.96-1.93 (m, 2H), 1.89-1.80 (m, 2H). ¹³C NMR (101 MHz, MeOD) δ 143.1 (CH), 142.7 (CH), 129.4 (CH), 112.9 (CH), 111.5 (CH), 101.6 (CH), 68.3 (CH), 55.0 (CH₃), 53.3 (CH₂), 52.3 (CH₂), 46.5 (CH₂), 43.2 (CH), 20.5 (CH₂). ESI-MS: measured m/z 270.00 [M+H]⁺. Purity by HPLC: 96.7% at 254 nm.

### Example 20:3-(2-((1S,4R)-2-Azabicyclo[2.2.1]Hept-5-En-2-Yl)Ethyl)-5-Methoxy-1H-Indole (16)

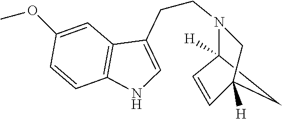 [SMILES: COc1ccc2[nH]cc(CCN3C[C@H]4C=C[C@@H]3C4)c2c1]

The title compound 3-(2-((1S,4R)-2-azabicyclo[2.2.1]hept-5-en-2-yl)ethyl)-5-methoxy-1H-indole (16), was prepared (3.5 mg) according to the protocol adopted for the synthesis of 15 and starting from 1-((1S,4R)-2-azabicyclo[2.2.1]hept-5-en-2-yl)-2-(5-methoxy-1H-indol-3-yl) ethan-1-one. ¹H NMR (400 MHz, MeOD) δ 7.22 (d, J=8.7 Hz, 1H), 7.04-6.98 (m, 2H), 6.76 (dd, J=8.8, 2.4 Hz, 1H), 6.48-6.42 (m, 1H), 6.12 (dd, J=5.7, 2.2 Hz, 1H), 4.09-4.03 (m, 1H), 3.83 (s, 3H), 3.20 (dd, J=9.0, 3.1 Hz, 1H), 3.05-2.93 (m, 2H), 2.93-2.84 (m, 1H), 2.81-2.70 (m, 1H), 2.62-2.49 (m, 1H), 1.73-1.63 (m, 2H), 1.54-1.48 (m, 1H). ¹³C NMR (101 MHZ, MeOD)δ 137.5 (CH), 130.3 (CH), 122.5 (CH), 111.5 (CH), 111.0 (CH), 99.9 (CH), 64.7 (CH), 55.6 (CH₂), 54.9 (CH₃), 52.0 (CH₂), 47.2 (CH₂), 43.3 (CH), 24.1 (CH₂). ESI-MS: measured m/z 269.00 [M+H]⁺. Purity by HPLC: 98% at 254 nm.

### Example 21: (2S,12R,12aS)-8-Methoxy-1,5,6,11,12,12a-Hexahydro-2,12-Methanopyrrolo[1′,2′: 1,2]Azepino[4,5-b]Indol-3 (2H)-One (17)

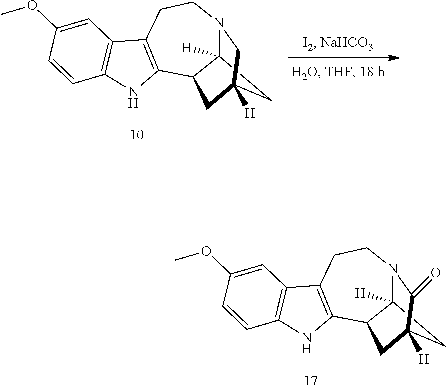

To a 25 mL round-bottom flask equipped with a inert atmosphere and in an ice bath, (2S,12R, 12aS)-8-methoxy-1,2,3,5,6,11,12,12a-octahydro-2,12-methanopyrrolo[1′,2′: 1,2]azepino[4,5-b]indole (100 mg, 328.07 μmol)(10) followed by a 5:4 mixture of THF/H₂O (2 mL, 0.07 M) and solid Sodium bicarbonate (151.59 mg, 1.80 mmol, 70.21 μL) were added. Iodine (249.80 mg, 984.21 μmol, 50.67 μL) was dissolved in THF (3 mL, 0.14 M), and the resulting solution was added dropwise over 10 min to the reaction mixture. Once the addition was finished, the ice bath was removed, and the solution was allowed to reach room temperature. The reaction was monitored by LC-MS, after over night, water and CH₂Cl₂ were added to dilute the reaction, in addition to a saturated solution of Na₂S₂O₃, and vigorous stirring was maintained for 5 min. The aqueous layer was extracted with CH₂Cl₂ (3×20 mL), the combined organic layer was dried over Na₂SO₄, and the solvent was removed by rotavapor. The crude product was purified by reverse phase column chromatography (30 gm; 0-100% ACN in water (0.1% Formic acid was added in both solvents)). The desired product (17) was obtained as a white solid (3.6 mg). ¹H NMR (400 MHZ, MeOD)δ 7.13 (d, J=8.7 Hz, 1H), 6.92 (d, J=2.4 Hz, 1H), 6.70 (dd, J=8.7, 2.4 Hz, 1H), 4.47-4.40 (m, 1H), 4.00-3.91 (m, 1H), 3.81 (s, 3H), 3.68-3.62 (m, 1H), 3.15-3.04 (m, 1H), 3.03-2.91 (m, 1H), 2.90-2.84 (m, 1H), 2.62-2.51 (m, 1H), 2.04-1.97 (m, 1H), 1.74-1.68 (m, 1H), 1.53-1.46 (m, 1H). ¹³C NMR (101 MHZ, MeOD)δ 110.5 (CH), 110.5 (CH), 99.5 (CH), 62.0 (CH), 54.9 (CH₃), 46. (CH), 42.9 (CH₂), 41.5 (CH), 39.7 (CH₂), 33.9 (CH₂), 20.7 (CH₂). ESI-MS: measured m/z 283.27 [M+H]⁺. Purity by HPLC: 96.0% at 254 nm.

### Example 22: (2S,12R,12aS)-8-Methoxy-5-Methyl-2,3,12,12a-Tetrahydro-1H-2,12-Methanobenzofuro[2,3-d]Pyrrolo[1,2-a]Azepine (18)

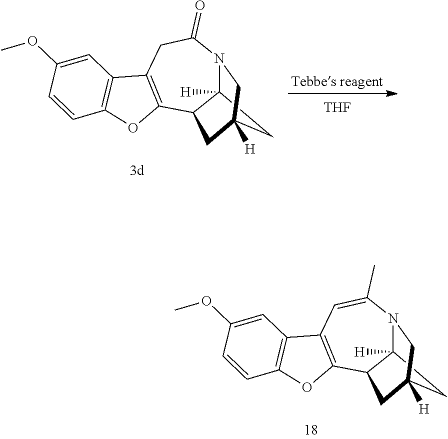

To a stirred solution of Tebbe Reagent, in toluene (0.5 M, 423.55 μL) at −60° C., added (2S,12R, 12aS)-8-methoxy-1,2,3,6,12,12a-hexahydro-5H-2,12-methanobenzofuro[2,3-d]pyrrolo[1,2-a]azepin-5-one (50 mg, 176.48 μmol)(3d) dissolved in THF, anhydrous (3 mL). The mixture was stirred at −60° C. for 3 hours, then brought to −20° C. for additional 40 min before quenching with saturated NaHCO₃. The reaction mixture was then filtered through celite by dissolving in EtOAc. Organic layer was dried on sodium sulfate, filtered. Filtrate was concentrated and purified by normal phase column chromatography (25 g; 20-100% EtOAc in Hexanes). The desired compound (18) was obtained (4.3 mg). ¹H NMR (400 MHZ, CDCl₃)δ 7.16 (d, J=8.8 Hz, 1H), 6.93 (d, J=2.6 Hz, 1H), 6.72 (dd, J=8.8, 2.6 Hz, 1H), 5.54-5.45 (m, 1H), 3.85-3.70 (m, 4H), 3.69-3.59 (m, 1H), 3.13-3.02 (m, 1H), 2.61-2.50 (m, 1H), 2.44-2.38 (m, 1H), 2.37-2.26 (m, 1H), 1.99-1.89 (m, 4H), 1.77-1.69 (m, 1H), 1.16-1.09 (m, 1H). 13C NMR (101 MHz, CDCl₃) δ 111.5 (CH), 110.8 (CH), 101.5 (CH), 96.2 (CH), 59.3 (CH₂), 58.7 (CH), 56.0 (CH₃), 46.5 (CH), 38.6 (CH₂), 35.1 (CH₂), 34.8 (CH), 22.0 (CH₃). ESI-MS: measured m/z 300.20 [M+H]⁺. Purity by HPLC: 99.4% at 254 nm.

### Example 23: Synthesis of (2S,12R,12aS)-5-Methyl-2,3,12,12a-Tetrahydro-1H-2,12-Methanobenzofuro[2,3-d]Pyrrolo[1,2-a]Azepin-8-Ol (19)

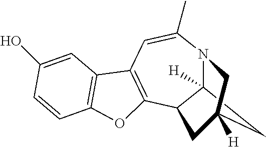

The title compound (2S,12R,12aS)-5-methyl-2,3,12,12a-tetrahydro-1H-2,12-methanobenzofuro[2,3-d]pyrrolo[1,2-a]azepin-8-ol (19), was prepared (4.5 mg) according to the protocol described in general procedure (IV) starting from (2S,12R, 12aS)-8-methoxy-5-methyl-2,3,12,12a-tetrahydro-1H-2,12-methanobenzofuro[2,3-d]pyrrolo[1,2-a]azepine (18). ¹H NMR (400 MHZ, CDCl₃)δ 7.21 (d, J=8.6 Hz, 1H), 6.99 (d, J=2.6 Hz, 1H), 6.73 (dd, J=8.6, 2.6 Hz, 1H), 5.55 (s, 1H), 3.87 (dd, J=11.3, 5.8 Hz, 1H), 3.75-3.69 (m, 1H), 3.16 (s, 1H), 2.64 (dd, J=7.1, 1.6 Hz, 1H), 2.52-2.47 (s, 1H), 2.45-2.38 (m, 1H), 2.05-1.99 (m, 4H), 1.84 (d, J=9.5 Hz, 1H), 1.25-1.24 (m, 1H). ESI-MS: measured m/z 268.20 [M+H]⁺. Purity by HPLC: 92.0% at 254 nm.

### Example 24: (2S,12R,12aS)-8-Methoxy-5-Methyl-2,3,5,6,12,12a-Hexahydro-1H-2,12-Methanobenzofuro[2,3-d]Pyrrolo[1,2-a]Azepine (20)

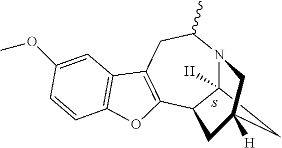

A 50 mL RBF was charged with (2S,12R,12aS)-8-methoxy-5-methyl-2,3,12,12a-tetrahydro-1H-2,12-methanobenzofuro[2,3-d]pyrrolo[1,2-a]azepine (32.3 mg, 114.80 μmol)(18) in DCM (2.5 mL) and MeOH (2.5 mL). Added Pd/C(12.22 mg, 114.80 μmol), degassed, and hydrogen balloon was attached continued to stir for overnight. Reaction mixture was passed through celite pad and washed the celite pad with MeOH. Concentrate the filtrate and the residue was purified by normal phase column chromatography running a mobile phase of 0-20% MeOH in DCM, and the product containing fractions were dried under reduced pressure to afford the desired product (20)(4.4 mg). ¹H NMR (400 MHZ, CDCl₃)δ 7.26 (d, J=8.8 Hz, 1H), 6.87 (d, J=2.6 Hz, 1H), 6.82 (dd, J=8.8, 2.6 Hz, 1H), 3.87 (s, 3H), 3.70-3.57 (m, 2H), 3.40-3.28 (m, 1H), 3.23-3.16 (m, 1H), 2.97-2.86 (m, 1H), 2.68-2.59 (m, 2H), 2.54-2.46 (m, 1H), 2.36-2.25 (m, 1H), 1.40-1.32 (m, 3H), 0.95-0.83 (m, 2H). ESI-MS: measured m/z 284.27 [M+H]⁺. Purity by HPLC: 99% at 254 nm.

### Example 25: ((2S,12R,12aS)-5-Methyl-2,3,5,6,12,12a-Hexahydro-1H-2,12-Methanobenzofuro[2,3-d]Pyrrolo[1,2-a]Azepin-8-Ol (21)

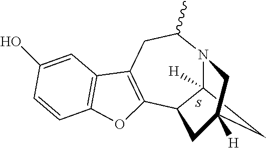

The title compound (2S,12R,12aS)-5-methyl-2,3,5,6,12,12a-hexahydro-1H-2,12-methanobenzofuro[2,3-d]pyrrolo[1,2-a]azepin-8-ol (21), was prepared (7.7 mg) according to the protocol described in general procedure (IV) starting from (2S,12R,12aS)-8-methoxy-5-methyl-2,3,5,6,12,12a-hexahydro-1H-2,12-methanobenzofuro[2,3-d]pyrrolo[1,2-a]azepine (20). ¹H NMR (400 MHZ, CDCl₃)δ 7.19 (d, J=8.7 Hz, 1H), 6.85 (d, J=2.4 Hz, 1H), 6.76 (dd, J=8.7, 2.5 Hz, 1H), 3.71 (s, 1H), 3.67-3.57 (m, 1H), 3.52 (s, 1H), 3.36-3.20 (m, 2H), 2.86 (dd, J=16.7, 11.6 Hz, 1H), 2.72-2.61 (m, 2H), 2.49 (dd, J=16.7, 2.8 Hz, 1H), 2.32 (tt, J=12.3, 3.7 Hz, 1H), 2.10-2.02 (m, 1H), 1.62-1.56 (m, 1H), 1.35 (d, J=6.6 Hz, 3H). (101 MHz, CDCl₃) δ 111.9 (CH), 110.9 (CH), 103.7 (CH), 63.9 (CH), 54.7 (CH), 50.8 (CH), 48.2 (CH₂), 39.2 (CH₂), 38.9 (CH₂), 37.4 (CH), 24.5 (CH₂), 21.4 (CH). ESI-MS: measured m/z 299.27 [M+H]⁺. Purity by HPLC: 97.7% at 254 nm.

### Example 26: (2S,12R,12aS)-8-Methoxy-5-Methyl-2,3,5,6,12,12a-Hexahydro-1H-2,12-Methanobenzofuro[2,3-d]Pyrrolo[1,2-a]Azepin-6-Ol (22)

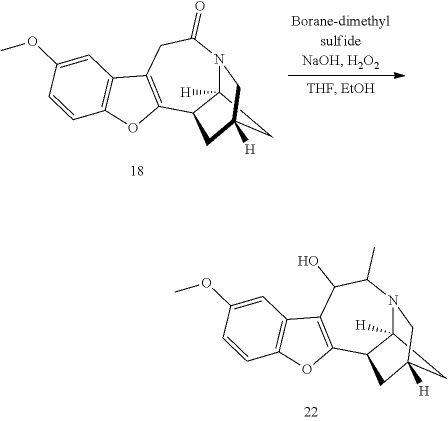

To a stirred solution of (2S,12R,12aS)-8-methoxy-5-methyl-2,3,12,12a-tetrahydro-1H-2,12-methanobenzofuro[2,3-d]pyrrolo[1,2-a]azepine (36.9 mg, 131.15 μmol)(18) in THF (3 mL), borane dimethyl sulfide complex in THF (2 M, 2.62 mmol, 1.31 mL) was added dropwise at 0° C. Then the reaction left to stir at room temperature for 48 hrs. Removed all the volatiles from the reaction mixture and added Ethanol (3 mL) and cooled 0° C. Added Sodium Hydroxide (104.92 mg, 2.62 mmol) and Hydrogen peroxide 35% (66.92 mg, 1.97 mmol, 60.83 μL) to the reaction mixture at the same temperature. The reaction left to stir at room temperature for an hour and quenched with water. The organic layer was extracted with DCM, concentrated. The residue was treated with 10% HCl in ether (1 mL) for an hour. Removed all the volatiles and residue was purified by normal phase column chromatography running a mobile phase of 20-100% EtOAc in Hexane, and the product containing fractions were dried under reduced pressure to afford the desired product (22)(4.4 mg). ¹H NMR (400 MHZ, MeOD)δ 7.47 (d, J=2.6 Hz, 1H), 7.19 (d, J=8.8 Hz, 1H), 6.79 (dd, J=8.8, 2.7 Hz, 1H), 5.15 (s, 0.4H), 4.85 (s, 0.6H), 3.84-3.79 (m, 3H), 3.63-3.58 (m, 1H), 3.39-3.34 (m, 1H), 3.32-3.27 (m, 1H), 3.23-3.17 (m, 1H), 2.58-2.51 (m, 2H), 2.30-2.20 (m, 1H), 1.81-1.74 (m, 1H), 1.53 (d, J=9.2 Hz, 1H), 1.40-1.36 (m, 3H), 1.29-1.22 (m, 1H). ¹³C NMR (101 MHZ, MeOD)δ 111.4 (CH), 109.8 (CH), 104.9 (CH), 68.4 (CH), 62.9 (CH), 60.0 (CH), 54.8 (CH₃), 48.3 (CH₂), 43.4 (CH), 39.4 (CH₂), 38.6 (CH₂), 38.0 (CH), 17.1 (CH₃). ESI-MS: measured m/z 300.20 [M+H]⁺. Purity by HPLC: 97.7% at 254 nm.

### Example 27:1-((2S,3aS,11bR)-8-Methoxy-1,2,3,3a,4,5,6,11b-Octahydrobenzofuro[2,3-d]Cyclopenta[b]Azepin-2-Yl) Ethan-1-Ol (23)

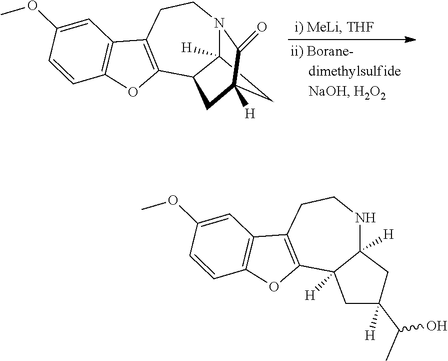

To a stirred solution of (2S,12R, 12aS)-8-methoxy-1,2,5,6,12,12a-hexahydro-3H-2,12-methanobenzofuro[2,3-d]pyrrolo[1,2-a]azepin-3-one (100 mg, 352.96 μmol) in THF (19.55 mL) at −78° C., added solution of Methyllithium solution (1.6 M, 1.06 mmol, 661.79 μL) dropwise manner under argon. The mixture was brought to room temperature and stirred for 30 minutes. Reaction was monitored by LCMS, after 30 minutes reaction brought to −78° C. and pH was adjusted to ˜8 with HCl in ether (1M solution), further diluted with water. Aqueous layer was extracted with EtOAc and dried over sodium sulfate. Filtered, filtrate was concentrated, and the residue was used in the next step without further purification. The residue was dissolved in THF (8.57 mL) and cooled to 0° C. Added borane dimethyl sulfide complex (54.00 mg, 710.86 μmol, 67.42 μL) in dropwise manner at 0° C. Then the reaction left to stir at room temperature for 90 min. Removed all the volatiles from the reaction mixture and dissolved in Ethanol (8.57 mL) and cooled at 0° C., followed by the slow addition of Sodium Hydroxide (3 M, 3.91 mmol, 1.30 mL) and Hydrogen peroxide 35% (362.70 mg, 3.20 mmol, 329.72 μL, 30% purity). After 2 hours, reaction was concentrated and extracted using water and EtOAc. Organic layer was dried over sodium sulfate and concentrated. The residue was purified by normal phase column chromatography running a mobile phase of 0-20% MeOH in DCM, and the product containing fractions were dried under reduced pressure to afford the desired product (23)(10.0 mg). ¹H NMR (400 MHZ, MeOD)δ 7.26 (d, J=8.8 Hz, 1H), 6.97 (d, J=2.3 Hz, 1H), 6.84 (dd, J=8.8, 2.3 Hz, 1H), 3.84 (s, 3H), 3.78-3.64 (m, 2H), 3.60-3.51 (m, 1H), 3.49-3.40 (m, 1H), 3.38-3.36 (m, 1H), 3.11-2.99 (m, 1H), 2.98-2.81 (m, 2H), 2.51-2.24 (m, 2H), 2.21-2.07 (m, 1H), 1.85-1.74 (m, 1H), 1.74-1.64 (m, 1H), 1.55-1.46 (m, 1H), 1.23-1.16 (m, 3H). 13C NMR (101 MHZ, MeOD)δ 111.8 (CH), 111.8 (CH), 110.4 (CH), 101.2 (CH), 101.2 (CH), 70.0 (CH), 70.0 (CH), 60.9 (CH), 60.8 (CH), 54.9 (CH), 48.5 (CH), 46.5 (CH₂), 46.4 (CH₂), 44.8 (CH), 44.6 (CH), 44.1 (CH), 43.9 (CH), 36.9 (CH₂), 34.9 (CH₂), 34.3 (CH₂), 32.2 (CH₂), 22.8 (CH₂), 22.7 (CH₂), 21.3 (CH₃), 20.9 (CH₃). ESI-MS: measured m/z 302.20 [M+H]. Purity by HPLC: 97.3% at 254 nm.

1. A compound of Formula (I): 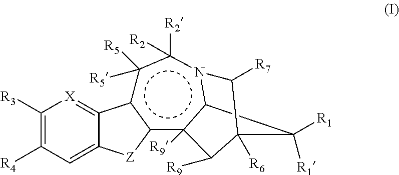
or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ, —C(═O)ORₐ, —CONHRₐ, —CONRₐRₐ, —CH₂ORₐ, or R₁ and R₁′ taken together with the atom to which they are attached form ═O, ═S, ═NH, ═NOH, or ═NORₐ;
R₂ and R₂′ are independently absent, hydrogen, deuterium, optionally substituted alkyl, optionally substituted aryl, optionally substituted heteroalkyl, —ORₐ-alkylene-ORₐ, —CH₂C(═O)ORₐ, —CH₂C(═O)NRₐRₐ or R₂ and R₂′ taken together with the atom to which they are attached form ═O, ═S, ═NH, ═NOH, and ═NORₐ;
each R₃ and R₄ is independently hydrogen, deuterium, —ORₐ, halogen, —NO₂, —CN, —NRₐRₐ, —SRₐ, —SORₐ, —SO₂Rₐ, —C(═O)ORₐ, —C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl, fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl, -optionally substituted heteroalkyl, —ORₐ, -alkylene-ORₐ—C(═O)ORₐ or —C(═O)NRₐRₐ;
R₇, R₉, and R₉′ are independently hydrogen, deuterium, optionally substituted alkyl, fluoro, —ORₐ, —C(═O)ORₐ, —CH₂ORₐ—CH₂SRₐ, —CH₂NRₐRₐ, aryl, heteroaryl, or cycloalkyl;
each Rₐ is independently hydrogen, alkyl, deuterated alkyl, alkenyl, alkylene-aryl, alkylene-cycloalkyl, aryl, or heteroaryl;
Z is O, S, or N(R₈), wherein R₅ is hydrogen, deuterium, alkyl, deuterated alkyl, —CH₂O—Rₐ, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ.

2. The compound of claim 1, wherein X is N.

3. The compound of claim 1, wherein X is C(R₃).

4. The compound of any one of claims 1-3, wherein Z is O.

5. The compound of any one of claims 1-3, wherein Z is N(R₈).

6. The compound of any one of claims 1-5, wherein R₁ and R₁′ are independently hydrogen, —ORₐ, or R₁ and R₁′ taken together with the atom to which they are attached form=O.

7. The compound of any one of claims 1-5, wherein R₁ and R₁′ are independently hydrogen, —OCH₃ or —OH.

8. The compound of any one of claims 1-7, wherein R₂ and R₂′ are independently hydrogen, optionally substituted alkyl, or -alkylene-ORₐ.

9. The compound of any one of claims 1-7, wherein R₂ and R₂′ are hydrogen.

10. The compound of any one of claims 1-7, wherein R₂ and R₂′ are independently hydrogen or —CH₂—OH.

11. The compound of any one of claims 1-10, wherein R₃ is —OH.

12. The compound of any one of claims 1-10, wherein R₃ is —OCH₃.

13. The compound of any one of claims 1-11, wherein R₄ is hydrogen.

14. The compound of any one of claims 1-11, wherein R₄ is —OH.

15. The compound of any one of claims 1-11, wherein R₄ is —OCH₃.

16. The compound of any one of claims 1-11, wherein R₃ and R₄ are —OH.

17. The compound of any one of claims 1-11, wherein R₃ and R₄ are —OCH₃.

18. The compound of any one of claims 1-17, wherein R₅ and R₅' are independently hydrogen, —ORₐ, or fluoro.

19. The compound of any one of claims 1-17, wherein R₅ and R₅' are independently hydrogen, or —OH.

20. The compound of any one of claims 1-17, wherein R₅ and R₅' are fluoro.

21. The compound of any one of claims 1-20, wherein R₆ is hydrogen.

22. The compound of any one of claims 1-21, wherein R₇ is hydrogen.

23. The compound of any one of claims 1-21, wherein R₇ is optionally substituted alkyl or —CH₂ORₐ.

24. The compound of any one of claims 1-21, wherein R₇ is —CH₂OH.

25. The compound of any one of claims 1-21, wherein Ry and R₉′ are hydrogen.

26. The compound of any one of claims 1-25, having the formula: 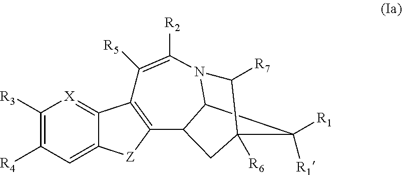
or a pharmaceutically acceptable salt, prodrug, or stereoisomer thereof.

27. The compound of claim 1, wherein the compound is selected from: 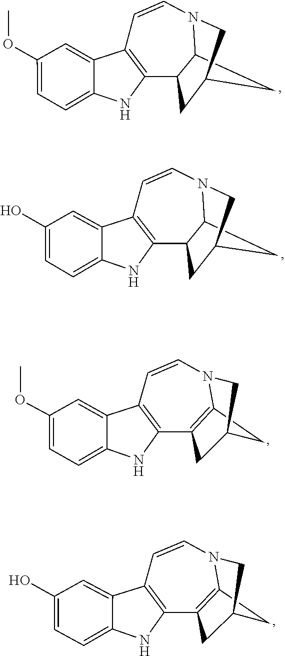 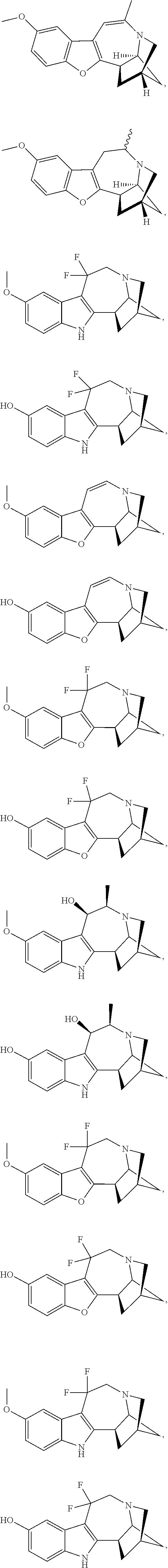 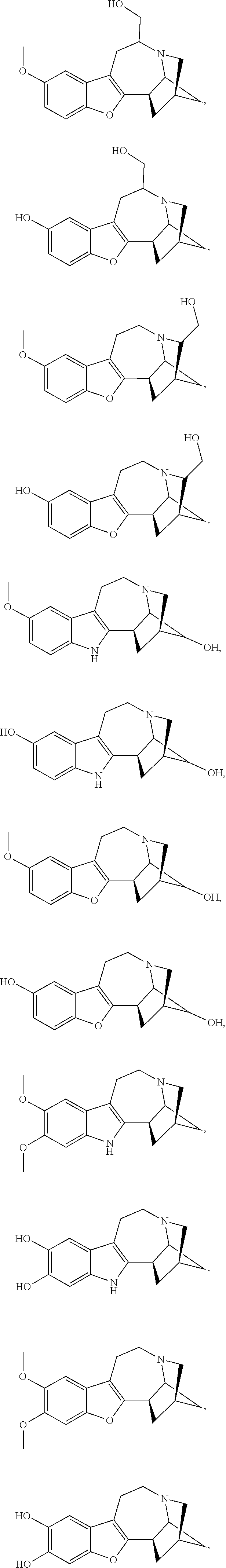
or a pharmaceutically acceptable salt, prodrug, or stereoisomer thereof.

28. A compound of Formula (II): 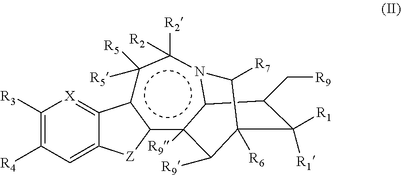
or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ, —C(═O)ORₐ, —CH₂ORₐ or R₁ and R₁′ taken together with the atom to which they are attached form ═NH, ═NOH, or ═NORₐ;
R₂ and R₂′ are independently absent, hydrogen, deuterium, optionally substituted alkyl, optionally substituted aryl, optionally substituted heteroalkyl, —ORₐ-alkylene-ORₐ, —CH₂C(═O)ORₐ, or —CH₂C(═O)NRₐRₐ;
each R₃ and R₄ is independently hydrogen, deuterium, —ORₐ, halogen, —NO₂, —CN, —NRₐRₐ, —SRₐ, —SORₐ, —SO₂Rₐ, —C(═O)ORₐ, —C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl, fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, -alkylene-ORₐ—C(═O)ORₐ—C(═O)NRₐRₐ;
R₇ is hydrogen, deuterium, optionally substituted alkyl (e.g., deuterated alkyl and halogenated alkyl), fluoro, —ORₐ, —C(═O)ORₐ, —CH₂ORₐ, —CH₂SRₐ, —CH₂NRₐRₐ, aryl, heteroaryl, or cycloalkyl;
R₉, R₉′ and R₉″ are independently hydrogen, deuterium, optionally substituted alkyl, fluoro, —ORₐ, —C(═O)ORₐ, —CH₂ORₐ—CH₂SRₐ, —CH₂NRₐRₐ, aryl, heteroaryl, or cycloalkyl each Rₐ is independently hydrogen, alkyl, deuterated alkyl, alkenyl, alkylene-aryl, alkylene-cycloalkyl, aryl, or heteroaryl;
Z is O, S, or N(R₈), wherein R₈ is hydrogen, deuterium, alkyl, deuterated alkyl, —CH₂O—Rₐ, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ,
wherein at least one of R₂, R₂′, R₅, R₅′, or R is not hydrogen; and
wherein the compound is not

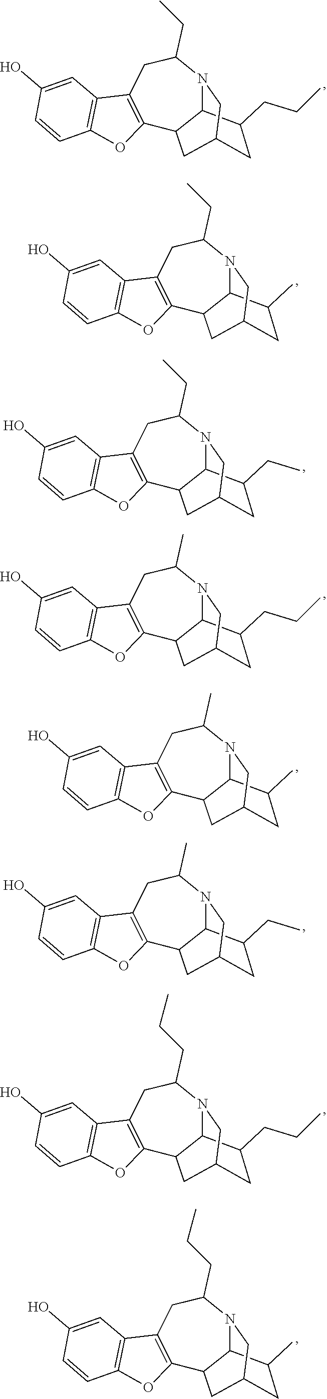

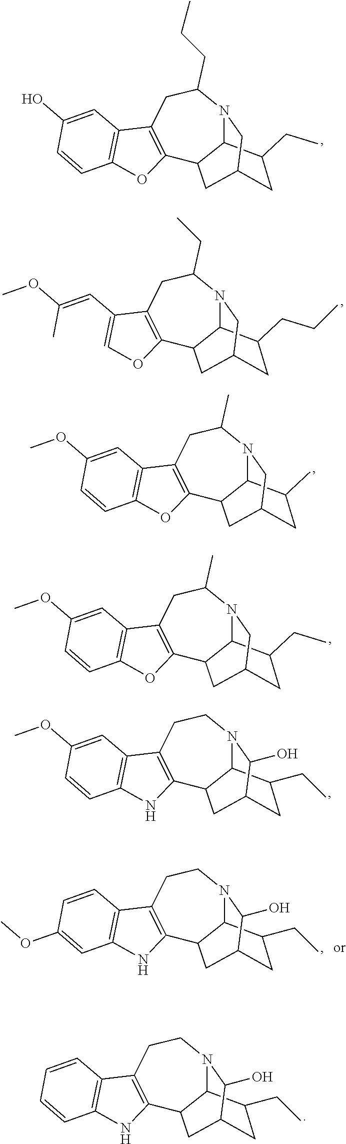

29. The compound of claim 28, wherein X is N.

30. The compound of claim 28, wherein X is C(R₃).

31. The compound of any one of claims 28-30, wherein Z is O.

32. The compound of any one of claims 28-30, wherein Z is N(R₈).

33. The compound of any one of claims 28-32, wherein R₁ and R₁′ are hydrogen.

34. The compound of any one of claims 28-33, wherein R₂ and R₂′ are independently hydrogen, optionally substituted alkyl, or alkylene-ORₐ.

35. The compound of any one of claims 28-33, wherein R₂ and R₂′ are hydrogen.

36. The compound of any one of claims 28-33, wherein R₂ and R₂′ are independently hydrogen or —CH₂—OH.

37. The compound of any one of claims 28-36, wherein R₃ is —OH.

38. The compound of any one of claims 28-36, wherein R₃ is —OCH₃.

39. The compound of any one of claims 28-38, wherein R₄ is hydrogen.

40. The compound of any one of claims 28-38, wherein R₄ is —OH.

41. The compound of any one of claims 28-38, wherein R₄ is —OCH₃.

42. The compound of any one of claims 28-38, wherein R₃ and R₄ are —OH.

43. The compound of any one of claims 28-38, wherein R₃ and R₄ are —OCH₃.

44. The compound of any one of claims 28-43, wherein R₅ and R₅' are independently hydrogen, —ORₐ, or fluoro.

45. The compound of any one of claims 28-43, wherein R₅ and R₅' are independently hydrogen, or —OH.

46. The compound of any one of claims 28-43, wherein R₅ and R₅' are fluoro.

47. The compound of any one of claims 28-46, wherein R₆ is hydrogen.

48. The compound of any one of claims 28-47, wherein R is hydrogen.

49. The compound of any one of claims 28-47, wherein R₇ is optionally substituted alkyl or —CH₂ORₐ.

50. The compound of any one of claims 28-47, wherein R₇ is —CH₂OH.

51. The compound of any one of claims 28-50, wherein R₉ is —CH₃.

52. The compound of any one of claims 28-50, wherein R₉′ and R₉″ are hydrogen.

53. The compound of any one of claims 28-52, having the formula: 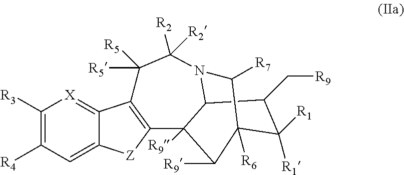
or a pharmaceutically acceptable salt, prodrug, or stereoisomer thereof.

54. The compound of claim 28, wherein the compound is selected from: 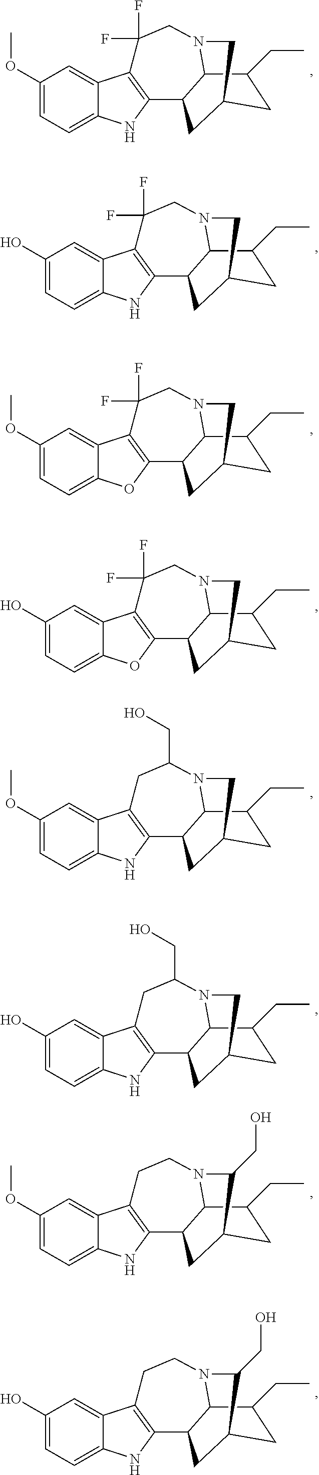
or a pharmaceutically acceptable salt, prodrug, or stereoisomer thereof.

55. A compound of Formula (III): 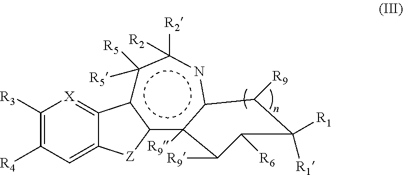
or a pharmaceutically acceptable salt, prodrug, or stereoisomer thereof, wherein:
X is C(R₃) or N;
R₁ and R₁′ are independently hydrogen, deuterium, fluoro, optionally substituted alkyl, —ORₐ, —NRₐRₐ—C(═O)ORₐ—CH₂ORₐ, or R₁ and R₁′ taken together with the atom to which they are attached form ═NH, ═NOH, or ═NORₐ;
R₂ and R₂′ are independently absent, hydrogen, deuterium, optionally substituted alkyl, optionally substituted aryl, optionally substituted heteroalkyl, —ORₐ-alkylene-ORₐ, —CH₂C(═O)ORₐ, —CH₂C(═O)NRₐRₐ, or R₂ and R₂′ taken together with the atom to which they are attached form ═NH, ═NOH, or ═NORₐ;
each R₃ and R₄ is independently hydrogen, deuterium, —ORₐ, halogen, —NO₂, —CN, —NRₐRₐ, —SRₐ, —C(═O)ORₐ—C(═O)NRₐRₐ, —OAc, —CH₂ORₐ, —CH₂SRₐ, or —CH₂NRₐRₐ;
R₅ and R₅' are independently absent, hydrogen, deuterium, optionally substituted alkyl, fluoro, —ORₐ, —C(═O)ORₐ, —CH₂ORₐ—CH₂SRₐ, —CH₂NRₐRₐ, aryl, heteroaryl, or cycloalkyl;
R₆ is hydrogen, deuterium, optionally substituted alkyl, optionally substituted aryl-optionally substituted heteroalkyl, —ORₐ, -alkylene-ORₐ—C(═O)ORₐ—C(═O)NRₐRₐ;
R₉, R₉′, and R₉″ are independently hydrogen, deuterium, optionally substituted alkyl, fluoro, —CN, —ORₐ, —C(═O)ORₐ, —C(═O)NRₐRₐ, —CH₂ORₐ—CH₂SRₐ, —CH₂NRₐRₐ, aryl, heteroaryl, or cycloalkyl
each Rₐ is independently hydrogen, alkyl, deuterated alkyl, alkenyl, alkylene-aryl, alkylene-cycloalkyl, aryl, or heteroaryl;
n is 0, 1 or 2; and
Z is O, S, or N(R₈), wherein R₈ is hydrogen, deuterium, alkyl, deuterated alkyl, —CH₂—OH, CH₂O—Rₐ, —CH₂SH, —CH₂S—Rₐ, —CH₂NRₐRₐ, —C(═O)ORₐ, —CONHRₐ, —P(═O)(OH)₂, —CH(Rₐ)OC(═O)ORₐ, —CH(Rₐ)OP(═O)(ORₐ)₂, —SO₂Rₐ, or —CONRₐRₐ.

56. The compound of claim 55, wherein X is N.

57. The compound of claim 55, wherein X is C(R₃).

58. The compound of any one of claims 55-57, wherein Z is O.

59. The compound of any one of claims 55-57, wherein Z is N(R₈).

60. The compound of any one of claims 55-57, wherein R₁ and R₁′ are hydrogen.

61. The compound of any one of claims 55-60, wherein R₂ and R₂′ are independently hydrogen, optionally substituted alkyl, or -alkylene-ORₐ.

62. The compound of any one of claims 55-60, wherein R₂ and R₂′ are hydrogen.

63. The compound of any one of claims 55-60, wherein R₂ and R₂′ are independently hydrogen or —CH₂—OH.

64. The compound of any one of claims 55-63, wherein R₃ is —OH.

65. The compound of any one of claims 55-63, wherein R₃ is —OCH₃.

66. The compound of any one of claims 55-65, wherein R₄ is hydrogen.

67. The compound of any one of claims 55-65, wherein R₄ is —OH.

68. The compound of any one of claims 55-65, wherein R₄ is —OCH₃.

69. The compound of any one of claims 55-65, wherein R₃ and R₄ are —OH.

70. The compound of any one of claims 55-65, wherein R₃ and R₄ are —OCH₃.

71. The compound of any one of claims 55-70, wherein R₅ and R₅' are independently hydrogen, —ORₐ, or fluoro.

72. The compound of any one of claims 55-70, wherein R₅ and R₅' are independently hydrogen, or —OH.

73. The compound of any one of claims 55-70, wherein R₅ and R₅' are fluoro.

74. The compound of any one of claims 55-73, wherein R₆ is hydrogen.

75. The compound of any one of claims 55-74, wherein R₉ is hydrogen.

76. The compound of any one of claims 55-75, wherein R₉′ and R₉″ are hydrogen.

77. The compound of claim 55, wherein the compound is 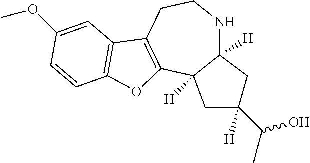 [SMILES: COc1ccc2oc3c(c2c1)CCN[C@H]1C[C@@H](C(C)O)C[C@@H]31]
or a pharmaceutically acceptable salt, prodrug, or stereoisomer thereof.

78. A pharmaceutical composition comprising any one of compounds of claims 1-77 or pharmaceutically acceptable salt, prodrug, or stereoisomer thereof and a pharmaceutically acceptable excipient.

79. A method of treating a disease or disorder in a patient in need thereof comprising administering a therapeutically effective amount of any one of compounds of claims 1-77 or the pharmaceutical composition of claim 78 to the patient.

80. The method of claim 79, wherein the diseases or disorder is alcoholism, substance abuse disorder, or opioid use disorder.
